(19) United States
(12) Patent Application Publication
(10) Pub. No.: US 2025/0218543 A1
(43) Pub. Date: Jul. 3, 2025

(54) Multi-objective Design Method for Confronting Two-pair primers

(71) Applicant: Tainan University of Technology, Tainan City (TW)

(72) Inventors: Cheng-Hong Yang, Tainan City (TW); Yu-Da Lin, Kaohsiung City (TW); Li-Yeh Chuang, Kaohsiung City (TW); Yu-Huei Cheng, Taichung City (TW); Yu-Jiun Weng, Kaohsiung City (TW)

(21) Appl. No.: 18/669,829

(22) Filed: May 21, 2024

(30) Foreign Application Priority Data
Dec. 27, 2023 (TW) 112151070

(51) Int. Cl. G16B 30/20

(52) U.S. Cl. CPC G16B 30/20

(57) ABSTRACT

A multi-objective design method for confronting two-pair primers is achieved by applying a calculation means to assess scores of at least one set of confronting two-pair primers according to a DNA template and a design objective so as to determine an optimal solution from the at least one set of confronting two-pair primers. The design objective includes a fourth objective and a ninth objective. The fourth objective is adapted for calculating an overall difference level by summing each differences level between a melting temperature of one of the primers and a preset melting temperature range. The ninth objective is adapted for calculating a product length difference level by summing each difference between a product length formed by one of primer pairs and a user-defined product length formed by a corresponding one of the primer pairs.

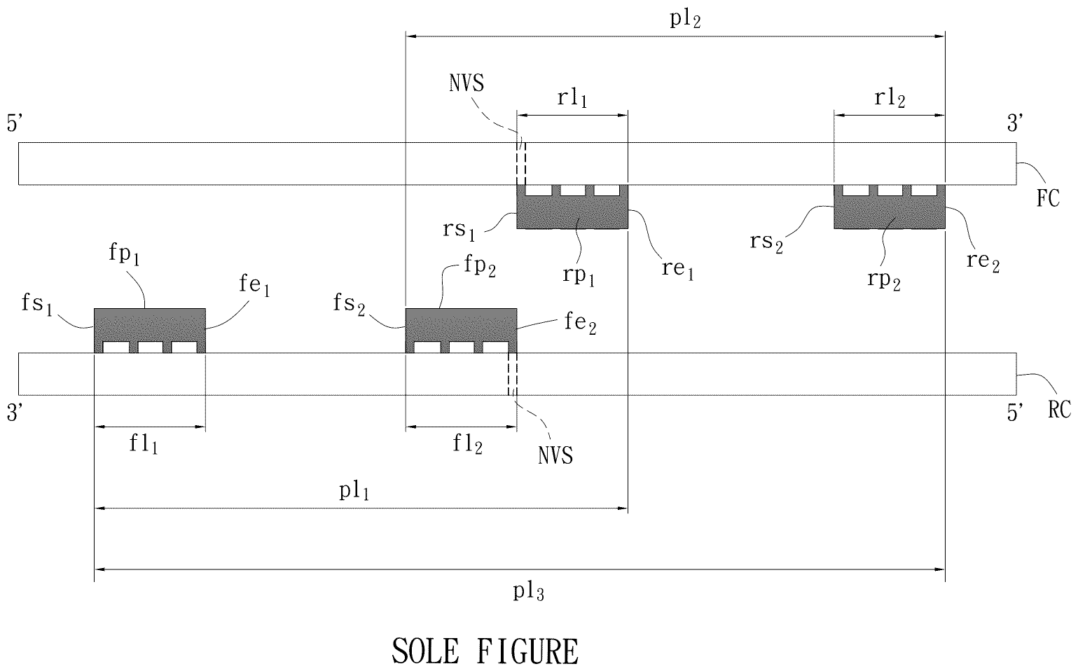

### CROSS REFERENCE TO RELATED APPLICATION

The application claims the benefit of Taiwan application serial No. 112151070, filed on Dec. 27, 2023, and the entire contents of which are incorporated herein by reference.

### BACKGROUND OF THE INVENTION

### 1. Field of the Invention

The present invention relates to a design method for confronting two-pair primers, more particularly, to a multi-objective design method for confronting two-pair primers.

### 2. Description of the Related Art

PCR (Polymerase Chain Reaction) is a chain reaction-based replication technique that can reproduce specific target gene sequences in large quantities. Primers, which are single-stranded oligonucleotides, serve as the starting point/segment for PCR replication. Since different primers may generate different PCR products and affect the corresponding performances of PCR, it is critical to use proper constraints/restricted for designing primers to determine corresponding primer design objectives to accurately reproduce/synthese target products (genes).

Genotyping is widely used in studying the association of diseases and cancer, and where SNP (Single-Nucleotide Polymorphism) is a common form of genomic variation occurring at a single nucleotide position within a gene, and this variation is prevalent in the human genome. Research on SNP genotyping typically involves molecular biology techniques such as PCR and gene sequencing to detect nucleotide polymorphisms in specific genomes, aiding in determining an individual's genotypic profile (i.e., the combination of carried allele genomes).

In conventional PCR techniques, a PCR-RFLP (polymerase chain reaction with restriction fragment length polymorphisms) technique is applied to reproduce target genes; however, PCR-RFLP requires longer digestion times (typically 2-3 hours) for restriction enzymes.

A PCR-CTPP (polymerase chain reaction with confronting two-pair primers) technique has been proposed as a replacement for the PCR-RFLP technique. The PCR-CTPP technique reduces the need for restriction enzymes and can be used for polymorphic genotyping to get proper and reliable results for genotyping in most nucleotide variation cases. Although the primer design for PCR-CTPP is similar to general traditional primer design (such as PCR-RFLP), PCR-CTPP must simultaneously consider the constraints of two sets of primers to establish corresponding primer design objectives/criteria based on restriction factors for synthesizing target products. Due to the design complexity for two sets of primers in PCR-CTPP, despite various common design objectives having been widely researched and proposed, there is still room for improvement in the conventional common design objectives to achieve more accurate synthesis of target products.

In light of the above, it is necessary to provide an improved primer design method to solve said conventional problems.

### SUMMARY OF THE INVENTION

To solve the above problems, it is an objective of the present invention to provide a multi-objective design method for confronting two-pair primers capable of improving the accuracy on the designed primer pairs to further improving the efficiency and accuracy for PCR.

As used herein, the term “a”, “an” or “one” for describing the number of the elements and members of the present invention is used for convenience, provides the general meaning of the scope of the present invention, and should be interpreted to include one or at least one.

Furthermore, unless explicitly indicated otherwise, the concept of a single component also includes the case of plural components.

As used herein, the term “length”, corresponding to “primer length”, “length of primer pair”, “product length” and the like, described in the full context of the present disclosure is measured by the number of bases or by any other unified unit commonly understood by skilled persons in the art.

All calculation means mentioned in the present disclosure have been disclosed in corresponding published documents, and are respectively listed as the following. It shall be noted, the contribution of the present invention is to propose design objective for improving the accuracy and efficiency of PCR-CTPP. Therefore, the calculation means are not limited to those mentioned in the present invention.
1. The Non-dominated Sorting Genetic Algorithm II (NSGAII) refers to a calculation means A of this invention.
2. The Multiple Objective Particle Swarm Optimization (MOPSO) refers to a calculation means B of this invention.
3. The Guided Population Archive Whale Optimization Algorithm (GPAWOA) refers to a calculation means C of this invention. The detailed contents of said Guided Population Archive Whale Optimization Algorithm can be found in the journal “A. Got, A. Moussaoui, D. Zouache, (2020). A guided population archive whale optimization algorithm for solving multiobjective optimization problems. Expert Systems with Applications, 141, 112972”, and all the contents of said journal are included in the present invention.

As used herein, the term “Pareto Chart”, which refers a Pareto optimization skill and can be represented as “Pareto Chart Analysis”, “Pareto Chart Tool”, “Pareto Optimization”, “Pareto Set” and the like, described in the full text of the present disclosure has a known definition in the art. When applying said Pareto optimization skill to access multiple solutions and only a single one of the multiple solutions is determined to be an optimal solution, the optimal solution dominates the others of the multiple solutions. When applying said Pareto skill to access multiple solutions and some of the multiple solutions are determined to be optimal solutions, those optimal solutions do not dominate to each other, and those optimal solutions each dominates the others of the multiple solutions.

As used herein, the term “computer” described in the full text of the present disclosure can contain at least one “processor”. The processor refers to one of various data processing apparatuses with specific functions and implemented in hardware or hardware and software, and can be configured to process analysis information and/or generate corresponding control information. In addition, a corresponding data receiving or transmission unit can be included to receive or transmit the required data. In addition, a corresponding database/storage unit (especially a non-transitory memory unit) can be included to store the required data. In particular, unless specifically excluded or contradicted, the processor can be a collection of multiple processors based on the distributed system architecture, which contains/represents the process, mechanism and results of information stream processed among the multiple processors.

A multi-objective design method for confronting two-pair primers according to the invention, is executed by a computer to execute the following steps. A DNA template fragment and a design objective is inputted. A calculation means is applied to assess scores of at least one set of confronting two-pair primers according to the DNA template and the design objective so as to determine an optimal solution from the at least one set of confronting two-pair primers. Wherein, the DNA template fragment includes a forward chain information and a reverse chain information. The forward chain information includes compositions of a forward chain and a nucleotide variant site located on the forward chain. The reverse chain information includes compositions of a reverse chain and a nucleotide variant site located on the reverse chain. Wherein, said one set of confronting two-pair primers includes a first forward primer, a first reverse primer, a second forward primer and a second reverse primer. The first forward primer has a first forward primer length. The first reverse primer has a first reverse primer length, and has a nucleotide variant site corresponding to the nucleotide variant site of the forward chain. The second forward primer has a second forward primer length, and has a nucleotide variant site corresponding to the nucleotide variant site of the reverse chain. The second reverse primer has a second reverse primer length. The first forward primer and the first reverse primer are defined to be a first primer pair having a first length. The second forward primer and the second reverse primer are defined to be a second primer pair having a second length. The first forward primer and the second reverse primer define a third length. Wherein, the design objective includes a fourth objective and a ninth objective.

The fourth objective is adapted for calculating an overall difference level by summing each differences level between a melting temperature of one of the primers and a preset melting temperature range, and is defined by the following formula.

$${f}_{4} = {Tm}_{dif} ( {fp}_{1} ) + {Tm}_{dif} ( {rp}_{1} ) + {Tm}_{dif} ( {fp}_{2} ) + {Tm}_{dif} ( {rp}_{2} ) ;$$

$${Tm}_{dif} ( primer ) = \{ \begin{matrix}{Tm}_{min} - {Tm}_{BM} ( primer ) , if {Tm}_{BM} ( primer ) < {Tm}_{min} \\ {Tm}_{BM} ( primer ) - {Tm}_{max} , if {Tm}_{BM} ( primer ) > {Tm}_{max} \\ 0 , if {Tm}_{min} \leq {Tm}_{BM} ( primer ) \leq {Tm}_{max}\end{matrix} ;$$

$${Tm}_{BM} ( primer ) = 81.5 + 166 \times ( log [ {Na}^{+} ] ) + 0.41 \times GC \% - 675 / {primer}_{LEN} ;$$

Wherein, f₄ is used for the fourth objective; Tm_{dif}(primer) is used for representing a difference level between a melting temperature of one of the primers and the preset melting temperature range; Tm_{BM}(primer) is used for representing the melting temperature for each one of the primers, and is calculated based on a Bolton and McCarthy formula; [Na⁺] is used for representing a molarity of Na ions of a solvent where a respective one primer is added; primer_{LEN} is used for representing a length of the respective one primer; Tmₘₐₓ and Tmₘᵢₙ are respectively used for representing a maximum value and a minimum value of the preset melting temperature range so as to mutually define a value range of the preset melting temperature range; fp₁, rp₁, fp₂ and rp₂ are respectively used for representing a first forward primer, a first reverse primer, a second forward primer and a second reverse primer in a corresponding one set of confronting two-pair primers; Tm_{dif}(fp₁), Tm_{dif}(rp₁), Tm_{dif}(fp₂) and Tm_{dif}(rp₂) are respectively used for representing the Tm_{dif}(primer) of the corresponding one of the first forward primer, the first reverse primer, the second forward primer and the second reverse primer.

The ninth objective is adapted for calculating a product length difference level by summing each difference between a product length formed by one of primer pairs and a user-defined product length formed by a corresponding one of the primer pairs, and is defined by the following formula.

$${f}_{9} = {product}_{{r}_{dif}} ( {pl}_{max} ) + {product}_{{r}_{dif}} ( {pl}_{min} ) + {product}_{{r}_{dif}} ( {pl}_{3} ) ;$$

$${product}_{{r}_{dif}} ( pl ) = \{ \begin{matrix}\vert {pl}^{r} - {pl}^{ur} \vert , & \vert {pl}^{r} - {pl}^{ur} \vert > t \\ 0 , & \vert {pl}^{r} - {pl}^{ur} \vert \leq t\end{matrix} ;$$

$$\{ \begin{matrix}{pl}_{sum} = {pl}_{max} + {pl}_{min} + {pl}_{3} \\ {pl}_{max}^{r} = {pl}_{max} \times \frac{100}{{pl}_{sum}} \\ {pl}_{min}^{r} = {pl}_{min} \times \frac{100}{{pl}_{sum}} \\ {pl}_{3}^{r} = {pl}_{3} \times \frac{100}{{pl}_{sum}}\end{matrix} and \{ \begin{matrix}{pl}_{sum}^{u} = {pl}_{max}^{u} + {pl}_{min}^{u} + {pl}_{3}^{u} \\ {pl}_{max}^{ur} = {pl}_{max}^{u} \times \frac{100}{{pl}_{sum}^{u}} \\ {pl}_{min}^{ur} = {pl}_{min}^{u} \times \frac{100}{{pl}_{sum}^{u}} \\ {pl}_{3}^{ur} = {pl}_{3}^{u} \times \frac{100}{{pl}_{sum}^{u}}\end{matrix} ;$$

Wherein, f₉ is used for the ninth objective; productᵣ_{_{dif}}(pl) is used for representing a definition on calculating a difference value for a ratio of the product length of a primer pair and a ratio of a user-defined product length; t is used for representing a user-defined difference value defining a predetermined difference ratio of the product length; pl^{r} is used for representing a ratio of the product length of one of the primer pairs; pl^{ur} is used for representing a ratio of the user-defined product length of a primer pair corresponding to pl^{r}; plₘₐₓ is used for representing a greater length between the first length and the second length, and is defined to be a maximum length; plₘᵢₙ is used for representing a smaller length between the first length and the second length, and is defined to be a minimum length; pl₃ is used for representing the third length which is larger than the maximum length and the minimum length; plₛᵤₘ is used for representing a sum length calculated by summing the first length, the second length and the third length; plₘₐₓ^{r} is used for representing a maximum length ratio calculated by dividing the maximum length over the sum length; plₘᵢₙ^{r} is used for representing a minimum length ratio calculated by dividing the minimum length over the sum length; pl₃^{r} is used for representing a third length ratio calculated by dividing the third length over the sum length; pl₃^{u} is used for representing a user-defined third length corresponding to the third length; plₘₐₓ^{u} and plₘᵢₙ^{u} are respectively used for representing a user-defined maximum length corresponding to the maximum length plₘₐₓ and a user-defined minimum length corresponding to the minimum length plₘᵢₙ, and both the user-defined maximum length and the user-defined minimum length are smaller than the user-defined third length; plₛᵤₘ^{u} is used for representing a user-defined sum length corresponding to the sum length plₛᵤₘ; plₘₐₓ^{ur} is used for representing a user-defined maximum length ratio calculated by dividing the user-defined maximum length over the user-defined sum length; plₘᵢₙ^{ur} is used for representing a user-defined minimum length ratio calculated by dividing the user-defined minimum length over the user-defined sum length; and pl₃^{ur} is used for representing a user-defined third length ratio calculated by dividing the user-defined third length over the user-defined sum length.

Therefore, by applying the proposed design objective adapted for reflecting the approximation/deviation extents to various interested characteristics or different preset conditions, the design results obtained by various calculation means overall have better performance than that obtained by conventional design objective. Further, the designed set of confronting two-pair primers, derived by said designed results, has improved accuracies to meet the requirements of the assessment indicators, and the corresponding PCR efficiency and accuracy are also improved accordingly.

In an example, the design objective further includes at least one of a first objective, a second objective, a third objective, a fifth objective, a sixth objective, a seventh objective, an eighth objective and a tenth objective. The first objective is used for calculating a difference level among the lengths of the primers. The second objective is used for calculating an overall difference level by summing each differences level between a proportion of nucleotides G and C in one of the primers and a preset GC proportion range. The third objective is used for calculating an overall stability of the primers. The fifth objective is used for calculating an overall difference level by summing each difference between the melting temperature of one of the primers and an average melting temperature of the primers. The sixth objective is used for calculating a dimer formation level for self-dimerization and cross-dimerization among the primers. The seventh objective is used for calculating an overall hairpin structure formation level of the primers. The eighth objective is used for calculating an overall specificity of the primers. The tenth objective is used for calculating an overall product length conformity level to reflect how many product lengths not less than a preset product length. Therefore, by applying at least one of the objectives adaptive for evaluating a specific characteristic, a corresponding characteristic of the designed result obtained by the method of the prevent invention has an inclination on meeting a corresponding objective requirement. That is, the method of the prevent invention improves the accuracy of the designed primers on meeting the corresponding objective requirement.

The first objective is defined by the following formula.

$${f}_{1} = \{ \begin{matrix}d 1 = 3 \\ if ( \vert {fl}_{1} - {rl}_{1} \vert \leq 3 ) , d 1 - 1 \\ if ( \vert {fl}_{2} - {rl}_{2} \vert \leq 3 ) , d 1 - 1 \\ if ( \vert {fl}_{1} - {rl}_{2} \vert \leq 3 ) , d 1 - 1\end{matrix} ;$$

Wherein, f₁ is used for representing the first objective; fl₁, fl₂, rl₁ and rl₂ are respectively used for representing the first forward primer length, the second forward primer length, the first reverse primer length and the second reverse primer length; d1 is an indicative value having a default value equal to 3 and is used for calculating the overall difference level among lengths of the primers; a total number of occurrences is initially defined to be zero, and in a condition that any to-be-evaluated length difference is not larger than 3, the total number of occurrences is accumulated by 1; the first objective is calculated by subtracting the total number of occurrences meeting said condition from the default value; said to-be-evaluated length differences are respectively defined by an absolute value calculated by deducting the first reverse primer length from the first forward primer length, an absolute value calculated by deducting the second reverse primer length from the second forward primer length, and an absolute value calculated by deducting the second reverse primer length from the first forward primer length. Therefore, by applying the first objective, the lengths of primer-pairs of the designed primers have the inclination on meeting the corresponding requirement of the first objective.

The second objective is defined by the following formula.

$${f}_{2} = GC {\%}_{range} = GC {\%}_{dif} ( {fp}_{1} ) + GC {\%}_{dif} ( {rp}_{1} ) + GC {\%}_{dif} ( {fp}_{2} ) + GC {\%}_{dif} ( {rp}_{2} ) ;$$

$$GC {\%}_{dif} ( primer ) = \{ \begin{matrix}GC {\%}_{min} - GC \% ( primer ) , if GC \% ( primer ) < GC {\%}_{min} \\ GC \% ( primer ) - GC {\%}_{max} , if GC \% ( primer ) > GC {\%}_{max} \\ 0 , if GC {\%}_{min} \leq GC \% ( primer ) \leq GC {\%}_{max}\end{matrix}$$

Wherein, f₂ is used for representing the second objective; GC %_{dif}(primer) is used for representing a difference level between a GC proportion in one of the primers and the preset GC proportion range; GC % (primer) is used for representing a GC proportion in one of the primers; GC % max and GC % min are respectively used for representing a maximum value and a minimum value of the preset GC proportion range so as to mutually define a value range of the preset GC proportion range; GC %_{dif}(fp₁), GC %_{dif}(rp₁), GC %_{dif}(fp₂) and GC %_{dif}(rp₂) are respectively used for representing the GC %_{dif}(primer) of the first forward primer, the first reverse primer, the second forward primer and the second reverse primer. Therefore, by applying the second objective, the GC proportions of the designed primers have the inclination on meeting the corresponding requirement of the second objective.

The third objective is defined by the following formula.

$${f}_{3} = \{ \begin{matrix}d 3 = 4 \\ if ( {3}^{′} of {fp}_{1} is G or C ) , d 3 - 1 \\ if ( {3}^{′} of {rp}_{1} is G or C ) , d 3 - 1 \\ if ( {3}^{′} of {fp}_{2} is G or C ) , d 3 - 1 \\ if ( {3}^{′} of {rp}_{2} is G or C ) , d 3 - 1\end{matrix} ;$$

Wherein, f₃ is used for representing the third objective; d3 is an indicative value having a default value equal to 4; a total number of occurrences is initially defined to be zero, and in a condition that the nucleotide type at the 3′ end of any one of the primers is G or C, the total number of occurrences is accumulated by 1; the third objective is calculated by subtracting the total number of occurrences meeting said condition from the default value. Therefore, by applying the third objective, the stabilities of the designed primers have the inclination on meeting the corresponding requirement of the third objective.

The fifth objective is defined by the following formula.

$${f}_{5} = {dif}_{Tm} ( {Tm}_{BM} ( {fp}_{1} ) , avgTm ) + {dif}_{Tm} ( {Tm}_{BM} ( {rp}_{1} ) , avgTm ) + {dif}_{Tm} ( {TM}_{BM} ( {fp}_{2} ) , avgTm ) + {dif}_{Tm} ( {Tm}_{BM} ( {rp}_{2} ) , avgTm ) ;$$

$$avgTm = [ {Tm}_{BM} ( {fp}_{1} ) + {Tm}_{BM} ( {rp}_{1} ) + {Tm}_{BM} ( {fp}_{2} ) + {TM}_{BM} ( {rp}_{2} ) ] \div 4 ;$$

Wherein, f₅ is used for representing the fifth objective; Tm_{BM}(fp₁), Tm_{BM}(rp₁), Tm_{BM}(fp₂) and Tm_{BM}(rp₂) each is used for representing the melting temperature of the corresponding one of the first forward primer, the first reverse primer, the second forward primer and the second reverse primer; avgTm is used for representing the average melting temperature. Therefore, by applying the fifth objective, the melting temperatures of the designed primers have the inclination on meeting the corresponding requirement of the fifth objective.

The sixth objective is defined by the following formula.

$${f}_{6} = Dimer ( {fp}_{1} , {rp}_{1} ) + Dimer ( {fp}_{1} , {rp}_{2} ) + Dimer ( {fp}_{2} , {rp}_{1} ) + Dimer ( {fp}_{2} , {rp}_{2} ) + Dimer ( {fp}_{1} , {fp}_{2} ) + Dimer ( {rp}_{1} , {rp}_{2} ) + Dimer ( {fp}_{1} , {fp}_{1} ) + Dimer ( {rp}_{1} , {rp}_{1} ) + Dimer ( {fp}_{2} , {fp}_{2} ) + Dimer ( {rp}_{2} , {rp}_{2} ) ;$$

$$Dimer ( {pm}_{a} , {pm}_{b} ) = {Dimer}_{num} ( {pm}_{a} , {pm}_{b} ) - {Dimer}_{user} ;$$

Wherein, f₆ is used for representing the sixth objective; Dimerₙᵤₘ(pmₐ, pm_{b}) is used for representing a corresponding binding quantity among identical primers or different primers; Dimerᵤₛₑᵣ is used for representing a user-defined primer biding quantity which is a value showing a binding quantity generated by nucleotides of any two of the primers; Dimer(pmₐ, pm_{b}) is used for representing a level on dimer formation between two of the primers, and is calculated by subtracting the user-defined primer biding quantity from a corresponding binding quantity between any two identical or different primers; Dimer(fp₁, rp₁) represents a level on dimer formation generated between the first forward primer and the first reverse primer; Dimer(fp₁, rp₂) represents a level on dimer formation generated between the first forward primer and the second reverse primer; Dimer(fp₂, rp₁) represents a level on dimer formation generated between the second forward primer and first reverse primer; Dimer(fp₂, rp₂) represents a level on dimer formation generated between the second forward primer and the second reverse primer; Dimer(fp₁, fp₂) represents a level on dimer formation generated between the first forward primer and the second forward primer; Dimer(rp₁, rp₂) represents a level on dimer formation generated between the first reverse primer and the second reverse primer;

Dimer(fp₁, fp₁) represents a level on dimer formation generated between two of the first forward primers; Dimer(rp₁, rp₁) represents a level on dimer formation generated between two of the first reverse primers; Dimer(fp₂, fp₂) represents a level on dimer formation generated between two of the second forward primers; Dimer(rp₂, rp₂) represents a level on dimer formation generated between two of the second reverse primers. Therefore, by applying the sixth objective, the dimer formations of the designed primers have the inclination on meeting the corresponding requirement of the sixth objective.

The seventh objective is defined by the following formula.

$${f}_{7} = Hairpin ( {fp}_{1} ) + Hairpin ( {rp}_{1} ) + Hairpin ( {fp}_{2} ) + Hairpin ( {rp}_{2} ) ;$$

$$Hairpin ( primer ) = {Hairpin}_{num} ( primer ) - {Hairpin}_{user} ;$$

Wherein, f is used for representing the seventh objective; Hairpinₙᵤₘ(primer) is used for representing a quantity of hairpin structure generated by a corresponding one of the primers; Hairpinᵤₛₑᵣ is used for representing a user-defined primer hairpin structure quantity which is a value showing a self-annealing quantity generated by nucleotides of a primer; Hairpin(primer) is used for representing a corresponding hairpin structure level of one of the primers, and is calculated by subtracting the user-defined primer hairpin structure quantity from a corresponding quantity of hairpin structure generated by a corresponding one of the primers; Hairpin(fp₁) represents a hairpin structure level of the first forward primer; Hairpin(rp₁) represents a hairpin structure level of the first reverse primer; Hairpin(fp₂) represents a hairpin structure level of the second forward primer; Hairpin(rp₂) represents a hairpin structure level of the second reverse primer. Therefore, by applying the seventh objective, the hairpin formations of the designed primers have the inclination on meeting the corresponding requirement of the seventh objective.

The eighth objective is defined by the following formula.

$${f}_{8} = {DNA}_{re} ( {fp}_{1} ) + {DNA}_{re} ( {rp}_{1} ) + {DNA}_{re} ( {fp}_{2} ) + {DNA}_{re} ( {rp}_{2} ) ;$$

Wherein, f₈ is used for representing the eighth objective; DNAᵣₑ(fp₁), DNAᵣₑ(rp₁), DNAᵣₑ(fp₂) and DNAᵣₑ(rp₂) are respectively used for representing a frequency re-appearing in the DNA template segment of the first forward primer, the first reverse primer, the second forward primer and the second reverse primer. Therefore, by applying the eighth objective, the specificities of the designed primers have the inclination on meeting the corresponding requirement of the eighth objective.

The tenth objective is defined by the following formula.

$${f}_{10} = \{ \begin{matrix}d 10 = 3 \\ if {pl}_{1} \geq 100 , d 10 - 1 \\ if {pl}_{2} \geq 100 , d 10 - 1 \\ if {pl}_{3} \geq 100 , d 10 - 1\end{matrix} ;$$

Wherein, f₁₀ is used for representing the tenth objective; d10 is an indicative value having a default value equal to 3 and is used for calculating the overall conformity level among product lengths; a total number of occurrences is initially defined to be zero, and in a condition that any to-be-evaluated product length is larger than or equal to 100, the a total number of occurrences is accumulated by 1; the tenth objective is calculated by subtracting the total number of occurrences meeting said condition from the default value; said to-be-evaluated product lengths are respectively defined by the first length, the second length and the third length. Therefore, by applying the tenth objective, the product lengths of the primer-pairs of the designed primers have the inclination on meeting the corresponding requirement of the tenth objective.

In an example, the design objective further includes the sixth objective and the seventh objective. Therefore, by additionally applying the sixth objective and the seventh objective, the dimer formations and the hairpin formations of the designed primers further have the inclination on meeting the corresponding requirement of the sixth and seventh objectives.

In an example, the design objective further includes the eighth objective. Therefore, by additionally applying the eighth objective, the specificities of the designed primers further have the inclination on meeting the requirement of the eighth objective.

In an example, the design objective further includes the first objective, the second objective, the third objective, the fifth objective, the sixth objective, the seventh objective, the eighth objective and the tenth objective. Therefore, by overall applying the first to tenth objectives, the overall characteristics of the design primers have the inclination on meeting the corresponding requirements of the first to tenth objectives.

In an example, in the calculation means, a Pareto Chart Analysis is applied to obtain the optimal solution. Therefore, the solutions derived by the Pareto Chart Analysis may be the same with or is close to the real optimal solution(s) for multi-objective problems.

In an example, the calculation means is a Guided Population Archive Whale Optimization Algorithm. Therefore, by using said specific calculation means and said specific objectives proposed by the present invention together, the overall performance of the design result derived thereby is better than that derived by other calculation means, so that said design result of the prevent invention may be the same with or is close to the real optimal solution(s), and the corresponding PCR efficiency and accuracy are also improved accordingly.

### BRIEF DESCRIPTION OF THE DRAWINGS

The present invention will become more fully understood from the detailed description given hereinafter and the accompanying drawings which are given by way of illustration only, and thus are not limitative of the present invention, and wherein:

The sole FIGURE is a schematic diagram of genetic segment/fragment to illustrate basic rules for designing confronting two-pair primers.

### DETAILED DESCRIPTION OF THE INVENTION

Referring to the sole FIGURE, the PCR-CTPP is designed based on providing a template including a specific nucleotide composition with a specific nucleotide variant. Based on said template including a forward chain FC, a reverse chain RC and a nucleotide variant site NVS for a DNA sequence, a first forward primer fp₁, a first reverse primer rp₁, a second forward primer fp₂ and a second reverse primer rp₂ should be correspondingly provided to act as the designed confronting two-pair primers. Said nucleotide variant sites NVS are located between the upstream side and downstream side of the forward chain FC and the reverse chain RC.

The first forward primer fp₁ is a short sense sequence corresponding a segment/fragment of the forward chain FC and extending in a direction from the upstream side of the forward chain FC (shown as the 5′ end of the forward chain FC in the sole FIGURE) to the downstream side of the forward chain FC (shown as the 3′ end of the forward chain FC in the sole FIGURE). The first forward primer fp₁ excludes the nucleotide variant site NVS and spaces a specific distance from the designated nucleotide variant site NVS.

Specifically, the first forward primer fp₁ has a first forward starting position fs₁ and a first forward ending position fe₁. The first forward starting position fs₁ is closer to the upstream side of the forward chain FC than the first forward ending position. The first forward ending position fe₁ is apart from the nucleotide variant site NVS at a specific distance. A first forward primer length fl₁ of the first forward primer fp₁ defines from the first forward starting position fs₁ to the first forward ending position fe₁.

The first reverse primer rp₁ is a short sense sequence corresponding a segment/fragment of the reverse chain RC and extending in a direction from the upstream side of the reverse chain RC (shown as the 3′ end of the reverse chain RC in the sole FIGURE) to the downstream side the reverse chain RC (shown as the 5′ end of the reverse chain RC in the sole FIGURE), and includes the nucleotide variant site NVS. Specifically, the first reverse primer rp₁ has a first reverse starting position rs₁ and a first reverse ending position re₁. The first reverse starting position rs₁ is closer to the upstream side of the reverse chain RC than the first reverse ending position re₁. A first reverse primer length rl₁ of the first reverse primer rp₁ defines from the first reverse starting position rs₁ to the first reverse ending position re₁. The first reverse primer rp₁ includes a nucleotide variant site NVS corresponding to the nucleotide variant site NVS of the forward chain FC. Preferably, the first reverse starting position rs₁ has the nucleotide variant site NVS.

The second forward primer fp₂ is a short sense sequence corresponding a segment/fragment of the forward chain FC and extending in a direction from the upstream side of the forward chain FC to the downstream side of the forward chain FC, and includes the nucleotide variant site NVS. Specifically, the second forward primer fp₂ has a second forward starting position fs₂ and a second forward ending position fe₂. The second forward starting position fs₂ is closer to the upstream side of the forward chain FC than the second forward ending position fe₂. A second forward primer length fl₂ of the second forward primer fp₂ is defined from the second forward starting position fs₂ to the second forward ending position fe₂. The second forward primer fp₂ includes a nucleotide variant site NVS corresponding to the nucleotide variant site NVS of the reverse chain RC. Preferably, the second forward ending position fe₂ has the nucleotide variant site NVS.

The second reverse primer rp₂ is a short sense sequence corresponding a segment/fragment of the reverse chain RC and extending in a direction from the upstream side of the reverse chain RC to the downstream side the reverse chain RC. The second reverse primer rp₂ excludes the nucleotide variant site NVS and spaces a specific distance from the designated nucleotide variant site NVS. Specifically, the second reverse primer rp₂ has a second reverse starting position rs₂ and a second reverse ending position re₂. The second reverse starting position rs₂ is closer to the upstream of the reverse chain RC than the second reverse ending position re₂. The nucleotide variant site NVS is closer to the upstream side of the reverse chain RC than the, and is apart from the second reverse primer rp₂. A second reverse primer length rl₂ of the second reverse primer rp₂ is defined from the second reverse starting position rs₂ to the second reverse ending position re₂.

The first forward primer fp₁ and the first reverse primer rp₁ are defined as the first primer pair having a first length pl₁. The second forward primer fp₂ and the second reverse primer rp₂ are defined as the second primer pair having a second length pl₂. A third length pl₃ is defined from the first forward primer fp₁ to the second reverse primer rp₂. Specifically, the first length pl₁ is defined from the first forward starting position fs₁ to the first reverse ending position re₁. The second length pl₂ is defined from the second forward starting position fs₂ to the second reverse ending position re₂. The third length pl₃ is defined from the first forward starting position fs₁ to the second reverse ending position re₂.

Based on the above-mentioned characteristics of the to-be-designed confronting two-pair primers (corresponding to the primers fp₁, fp₂, rp₁, rp₂), this invention provides an optimized design objective/constraints including ten objectives described as the following.

A first objective f₁ is provided. The first objective f₁ is adapted for calculating/evaluating a difference level among the lengths of the primers, and is defined by the following formula.

$${f}_{1} = {primer}_{LENdif} = \{ \begin{matrix}d 1 = 3 \\ if ( \vert {fl}_{1} - {rl}_{1} \vert \leq 3 ) , d 1 - 1 \\ if ( \vert {fl}_{2} - {rl}_{2} \vert \leq 3 ) , d 1 - 1 \\ if ( \vert {fl}_{1} - {rl}_{2} \vert \leq 3 ) , d 1 - 1\end{matrix} ;$$

In the formula of the first objective f₁, as the above-mentioned contents and referring to the sole FIGURE, fl₁, fl₂, rl₁ and rl₂ are respectively used for representing the first forward primer length, the second forward primer length, the first reverse primer length and the second reverse primer length; primer_{LENdif} is used for representing an indicator (lower is better) of the overall difference level among lengths of the primers (corresponding to the primers fp₁, rp₁, fp₂ and rp₂ as shown in the sole FIGURE) d1 is an indicative value having a default value equal to 3 and is used for calculating the overall difference level among lengths of the primers; a total number of occurrences is initially defined to be zero, and in a condition that any to-be-evaluated length difference is not larger than 3, the total number of occurrences is accumulated by 1; the first objective f₁/indicative value d1 is calculated/updated by subtracting a total number of occurrences meeting said condition from the default value; said to-be-evaluated length differences are respectively defined by an absolute value calculated by deducting the first reverse primer length rl₁ from the first forward primer length fl₁, an absolute value calculated by deducting the second reverse primer length rl₂ from the second forward primer length fl₂, and an absolute value calculated by deducting the second reverse primer length rl₂ from the first forward primer length fl₁.

For example, if all said to-be-evaluated length differences are larger than 3, the indicative value d1 is equal to 3. If only one of said to-be-evaluated length differences is not larger than 3, the indicative value d1 is equal to 2. If there are two of said to-be-evaluated length differences are not larger than 3, the indicative value d1 is equal to 1. If all said to-be-evaluated length differences are not larger than 3, the indicative value d1 is equal to 0.

A second objective f₂ is provided. The second objective f₂ is adapted for calculating/evaluating an overall difference level by summing each difference level between a proportion of nucleotides G and C (denoted as “GC proportion” hereinafter) in one of the primers and a preset GC proportion range, and is defined by the following formula.

$${f}_{2} = GC {\%}_{range} = GC {\%}_{dif} ( {fp}_{1} ) + GC {\%}_{dif} ( {rp}_{1} ) + GC {\%}_{dif} ( {fp}_{2} ) + GC {\%}_{dif} ( {rp}_{2} ) ;$$

$$GC {\%}_{dif} ( primer ) = \{ \begin{matrix}GC {\%}_{min} - GC \% ( primer ) , if GC \% ( primer ) < GC {\%}_{min} \\ GC \% ( primer ) - GC {\%}_{max} , if GC \% ( primer ) > GC {\%}_{max} \\ 0 , if GC {\%}_{min} \leq GC \% ( primer ) \leq GC {\%}_{max}\end{matrix} ;$$

In the formula of the second objective f₂, GC % range is used for representing an indicator (lower is better) derived by summing each difference level calculated from the GC proportion in each primer and the preset GC proportion range; GC %_{dif}(primer) is used for representing a difference level between a GC proportion in one of the primers (corresponding to the primers fp₁, rp₁, fp₂ and rp₂ as shown in the sole FIGURE) and the preset GC proportion range; GC % (primer) is used for representing a GC proportion in one of the primers; GC % max and GC % min are respectively used for representing a maximum value and a minimum value of the preset GC proportion range so as to mutually define a value range of the preset GC proportion range; GC %_{dif}(fp₁), GC %_{dif}(rp₁), GC %_{dif}(fp₂) and GC %_{dif}(rp₂) are respectively used for representing the GC %_{dif}(primer) of the first forward primer fp₁, the first reverse primer rp₁, the second forward primer fp₂ and the second reverse primer rp₂.

Particularly, in the second objective f₂, a maximum GC proportion (GC %ₘₐₓ) and a minimum GC proportion (GC %ₘᵢₙ) of said preset GC proportion range can be determined by characteristics of a PCR target. In an instance, the maximum GC proportion is 80% and the minimum GC proportion is 20%. Especially, for general cases, the maximum GC proportion can be set to not exceeding 80%, and the minimum GC proportion can be set to not less than 20%. However, it should be noted, the present invention is not limited to the numerical values mentioned above.

A third objective f₃ is provided. The third objective f₃ is adapted for calculating/evaluating an overall stability of the primers, and is defined by the following formula.

$${f}_{3} = {GC}_{clamp} = \{ \begin{matrix}d 3 = 4 \\ if ( {3}^{′} of {fp}_{1} is G or C ) , d 3 - 1 \\ if ( {3}^{′} of {rp}_{1} is G or C ) , d 3 - 1 \\ if ( {3}^{′} of {fp}_{2} is G or C ) , d 3 - 1 \\ if ( {3}^{′} of {rp}_{2} is G or C ) , d 3 - 1\end{matrix} ;$$

The third objective f₃ is defined based on the characteristic of the stability of each primer, and the stability is enhanced when the nucleotide type at the 3′ end of each primer is G or C. In the formula of the third objective f₃, GC_{clamp} is used for representing an indicator (lower is better) of the overall stability of the primers; d3 is an indicative value having a default value equal to 4; a total number of occurrences is initially defined to be zero, and in a condition that the nucleotide type at the 3′ end of any one of the primers is G or C, the total number of occurrences is accumulated by 1; the third objective f₃/indicative value d3 is calculated/updated by subtracting a total number of occurrences meeting said condition from the default value.

For example, if the nucleotide type at the 3′ end of all the primers are not G or C, the indicative value d3 is equal to 4. If only one of said primers has G or C nucleotide at its 3′ end, the indicative value d3 is equal to 3. If two of said primers respectively have G or C nucleotide at their 3′ end, the indicative value d3 is equal to 2. If three of said primers respectively have G or C nucleotide at their 3′ end, the indicative value d3 is equal to 1. If all of said primers respectively have G or C nucleotide at their 3′ end, the indicative value d3 is equal to 0.

A fourth objective f₄ is provided. The fourth objective f₄ is adapted for calculating/evaluating an overall difference level by summing each difference level between a melting temperature of one of the primers and a preset melting temperature range, and is defined as the following formula.

$${f}_{4} = {Tm}_{range} = {Tm}_{dif} ( {fp}_{1} ) + {Tm}_{dif} ( {rp}_{1} ) + {Tm}_{dif} ( {fp}_{2} ) + {Tm}_{dif} ( {rp}_{2} ) ;$$

$${Tm}_{dif} ( primer ) = \{ \begin{matrix}{Tm}_{min} - {Tm}_{BM} ( primer ) , if {Tm}_{BM} ( primer ) < {Tm}_{min} \\ {Tm}_{BM} ( primer ) - {Tm}_{max} , if {Tm}_{BM} ( primer ) > {Tm}_{max} \\ 0 , if {Tm}_{min} \leq {Tm}_{BM} ( primer ) \leq {Tm}_{max}\end{matrix} ;$$

$${Tm}_{BM} ( primer ) = 81.5 + 166 \times ( log [ {Na}^{+} ] ) + 0.41 \times GC \% - 675 / {primer}_{LEN} ;$$

In the formula of the fourth objective f₄, Tm_{range} is used for representing an indicator (lower is better) derived by summing each difference level calculated from the melting temperature of each primer and the preset melting temperature range; Tm_{dif}(primer) is used for representing a difference level between a melting temperature of one of the primers (corresponding to the primers fp₁, rp₁, fp₂ and rp₂ as shown in the sole FIGURE) and the preset melting temperature range; Tm_{BM}(primer) is used for representing the melting temperature for each one of the primers, and is calculated based on the Bolton and McCarthy formula; [Na⁺] represents a molarity of Na ions of a solvent where a respective one primer is added; primer_{LEN} represents a length of the respective one primer; Tmₘₐₓ and Tmₘᵢₙ are respectively used for representing a maximum value and a minimum value of the preset melting temperature range so as to mutually define a value range of the preset melting temperature range; Tm_{dif}(fp₁), Tm_{dif}(rp₁), Tm_{dif}(fp₂) and Tm_{dif}(rp₂) are respectively used for representing the Tm_{dif}(primer) of the corresponding one of the first forward primer fp₁, the first reverse primer rp₁, the second forward primer fp₂, and the second reverse primer rp₂.

Particularly, in the fourth objective f₄, a maximum melting temperature (Tmₘₐₓ) and a minimum melting temperature (Tmₘᵢₙ) of said preset melting temperature range can be determined by characteristics of a PCR target. In an instance, the maximum melting temperature is 62° C., and the minimum melting temperature is 45° C. Especially, for general cases, the maximum melting temperature can be set to not exceeding 80° C., and the minimum melting temperature can be set to not less than 50° C. However, it should be noted, the present invention is not limited to the numerical values mentioned above.

A fifth objective f₅ is provided. The fifth objective f₅ is adapted for calculating/evaluating an overall difference level by summing each difference between the melting temperature of one of the primers and an average melting temperature of the primers, and is defined by the following formula.

$${f}_{5} = {avgTm}_{dif} = {dif}_{Tm} ( {Tm}_{BM} ( {fp}_{1} ) , avgTm ) + {dif}_{Tm} ( {Tm}_{BM} ( {rp}_{1} ) , avgTm ) + {dif}_{Tm} ( {TM}_{BM} ( {fp}_{2} ) , avgTm ) + {dif}_{Tm} ( {Tm}_{BM} ( {rp}_{2} ) , avgTm ) ;$$

$$avgTm = [ {Tm}_{BM} ( {fp}_{1} ) + {Tm}_{BM} ( {rp}_{1} ) + {Tm}_{BM} ( {fp}_{2} ) + {TM}_{BM} ( {rp}_{2} ) ] \div 4 ;$$

In the formula of the fifth objective f₅, avgTm_{dif} is used for representing an indicator (lower is better) derived by summing differences calculated from the melting temperature of each primer and the average melting temperature; Tm_{BM}(fp₁), Tm_{BM}(rp₁), Tm_{BM}(fp₂) and Tm_{BM}(rp₂) each is used for representing the melting temperature of the corresponding one of the first forward primer fp₁, the first reverse primer rp₁, the second forward primer fp₂ and the second reverse primer rp₂; avgTm is used for representing the average melting temperature.

A sixth objective f₆ is provided. The sixth objective f₆ is adapted for calculating/evaluating a dimer formation level/extent where the dimers are generated by phenomena of self-dimerization or cross-dimerization among the primers, and is defined by the following formula.

$${f}_{6} = Dimer ( {fp}_{1} , {rp}_{1} ) + Dimer ( {fp}_{1} , {rp}_{2} ) + Dimer ( {fp}_{2} , {rp}_{1} ) + Dimer ( {fp}_{2} , {rp}_{2} ) + Dimer ( {fp}_{1} , {fp}_{2} ) + Dimer ( {rp}_{1} , {rp}_{2} ) + Dimer ( {fp}_{1} , {fp}_{1} ) + Dimer ( {rp}_{1} , {rp}_{1} ) + Dimer ( {fp}_{2} , {fp}_{2} ) + Dimer ( {rp}_{2} , {rp}_{2} ) ;$$

$$Dimer ( {pm}_{a} , {pm}_{b} ) = {Dimer}_{num} ( {pm}_{a} , {pm}_{b} ) - {Dimer}_{user} ;$$

In the formula of the sixth objective f₆, Dimer is used for representing an indicator (lower is better) derived by summing differences calculated from biding quantities among the primers and a user-defined primer biding quantity; Dimerₙᵤₘ(pmₐ, pm_{b}) is used for representing a corresponding binding quantity among identical primers or different primers; Dimerᵤₛₑᵣ is used for representing a user-defined primer biding quantity which is a value (especially acting as a maximum acceptable extent) defined by a user to show a binding quantity generated by nucleotides of any two of the primers; Dimer(pmₐ, pm_{b}) is used for representing a level on dimer formation between two of the primers, and is calculated by subtracting the user-defined primer biding quantity from a corresponding binding quantity between any two identical or different primers; Dimer(fp₁, rp₁) represents a level on dimer formation generated between the first forward primer fp₁ and the first reverse primer rp₁; Dimer(fp₁, rp₂) represents a level on dimer formation generated between the first forward primer fp₁ and the second reverse primer rp₂; Dimer(fp₂, rp₁) represents a level on dimer formation generated between the second forward primer fp₂ and first reverse primer rp₁; Dimer(fp₂, rp₂) represents a level on dimer formation generated between the second forward primer fp₂ and the second reverse primer rp₂; Dimer(fp₁, fp₂) represents a level on dimer formation generated between the first forward primer fp₁ and the second forward primer fp₂; Dimer(rp₁, rp₂) represents a level on dimer formation generated between the first reverse primer rp₁ and the second reverse primer rp₂; Dimer(fp₁, fp₁) represents a level on dimer formation generated between two of the first forward primers fp₁; Dimer(rp₁, rp₁) represents a level on dimer formation generated between two of the first reverse primers rp₁; Dimer(fp₂, fp₂) represents a level on dimer formation generated between two of the second forward primers fp₂; Dimer(rp₂, rp₂) represents a level on dimer formation generated between two of the second reverse primers rp₂.

A seventh objective f₇ is provided. The seventh objective f₇ is adapted for calculating/evaluating an overall hairpin structure formation level/extent of the primers, and is defined by the following formula.

$${f}_{7} = Hairpin = Hairpin ( {fp}_{1} ) + Hairpin ( {rp}_{1} ) + Hairpin ( {fp}_{2} ) + Hairpin ( {rp}_{2} ) ;$$

$$Hairpin ( primer ) = {Hairpin}_{num} ( primer ) - {Hairpin}_{user} ;$$

In the formula of the seventh objective f₇, Hairpin is used for representing an indicator (lower is better) to reflect the overall hairpin structure formation level for the primers; Hairpinₙᵤₘ(primer) is used for representing a quantity of hairpin structure generated by a corresponding one of the primers; Hairpinᵤₛₑᵣ is used for representing a user-defined primer hairpin structure quantity which is a value (especially acting as a maximum acceptable extent) defined by a user to show a self-annealing quantity generated by nucleotides of a primer; Hairpin(primer) is used for representing a corresponding hairpin structure level of one of the primers, and is calculated by subtracting the user-defined primer hairpin structure quantity from a corresponding quantity of hairpin structure generated by a corresponding one of the primers; Hairpin(fp₁) represents a hairpin structure level of the first forward primer fp₁; Hairpin(rp₁) represents a hairpin structure level of the first reverse primer rp₁; Hairpin(fp₂) represents a hairpin structure level of the second forward primer fp₂; Hairpin(rp₂) represents a hairpin structure level of the second reverse primer rp₂.

An eighth objective fs is provided. The eighth objective f₈ is adapted for calculating/evaluating an overall specificity of the primers, and is defined by the following formula.

$${f}_{8} = Specificity = {DNA}_{re} ( {fp}_{1} ) + {DNA}_{re} ( {rp}_{1} ) + {DNA}_{re} ( {fp}_{2} ) + {DNA}_{re} ( {rp}_{2} ) ;$$

In the formula of the eighth objective f₈, Specificity is used for representing an indicator (lower is better) to reflect the overall specificity of a confronting two-pair primers; DNAᵣₑ(fp₁), DNAᵣₑ(rp₁), DNAᵣₑ(fp₂) and DNAᵣₑ(rp₂) are respectively used for representing a frequency re-appearing in the DNA template segment/fragment of the first forward primer fp₁, the first reverse primer rp₁, the second forward primer fp₂ and the second reverse primer rp₂.

A ninth objective f₉ is provided. The ninth objective f₉ is adapted for calculating/evaluating a product length difference level by summing each difference between a product length formed by one of the primer pairs and a user-defined product length formed by a corresponding one of the primer pairs, and is defined by the following formula.

$${f}_{9} = {product}_{ratio} = {product}_{{r}_{dif}} ( {pl}_{max} ) + {product}_{{r}_{dif}} ( {pl}_{min} ) + {product}_{{r}_{dif}} ( {pl}_{3} ) ;$$

$${product}_{{r}_{dif}} ( pl ) = \{ \begin{matrix}\vert {pl}^{r} - {pl}^{ur} \vert & , & \vert {pl}^{r} - {pl}^{ur} \vert > t \\ 0 & , & \vert {pl}^{r} - {pl}^{ur} \vert \leq t\end{matrix} ;$$

$$\{ \begin{matrix}{pl}_{sum} = {pl}_{max} + {pl}_{min} + {pl}_{3} \\ {pl}_{max}^{r} = {pl}_{max} \times \frac{100}{{pl}_{sum}} \\ {pl}_{min}^{r} = {pl}_{min} \times \frac{100}{{pl}_{sum}} \\ {pl}_{3}^{r} = {pl}_{3} \times \frac{100}{{pl}_{sum}}\end{matrix} and \{ \begin{matrix}{pl}_{sum}^{u} = {pl}_{max}^{u} + {pl}_{min}^{u} + {pl}_{3}^{u} \\ {pl}_{max}^{ur} = {pl}_{max}^{u} \times \frac{100}{{pl}_{sum}^{u}} \\ {pl}_{min}^{ur} = {pl}_{min}^{u} \times \frac{100}{{pl}_{sum}^{u}} \\ {pl}_{3}^{ur} = {pl}_{3}^{u} \times \frac{100}{{pl}_{sum}^{u}}\end{matrix} ;$$

In the formula of the ninth objective f₉, productᵣₐₜᵢₒ is used for representing an indicator (lower is better) of overall difference level among product lengths of primer pairs and user-defined product lengths; productᵣ_{_{dif}}(pl) is used for representing a definition on calculating a difference value for a ratio of the product length of a primer pair and a ratio of a user-defined product length of a corresponding one primer pair; t is used for representing a predetermined/user-defined difference value defining an acceptable difference ratio of the product length; pl^{r} is used for representing a ratio of the product length of one of primer pairs; pl^{ur} is used for representing a ratio of the user-defined product length of a primer pair corresponding to pl^{r}; plₘₐₓ is used for representing a greater length between the first length pl₁ and the second length pl₂, and is defined as a maximum length; plₘᵢₙ is used for representing a smaller length between the first length pl₁ and the second length pl₂, and is defined as a minimum length; pl₃ is used for representing the third length pl₃ larger than the maximum length and the minimum length; plₛᵤₘ is used for representing a sum length calculated by summing the first length pl₁, the second length pl₂ and the third length pl₃; plₘₐₓ^{r} is used for representing a maximum length ratio calculated by dividing the maximum length over the sum length; plₘᵢₙ^{r} is used for representing a minimum length ratio calculated by dividing the minimum length over the sum length; pl₃^{r} is used for representing a third length ratio calculated by dividing the third length over the sum length; ply is used for representing a user-defined third length corresponding to the third length pl₃; plₘₐₓ^{u} and plₘᵢₙ^{u} are respectively used for representing a user-defined maximum length corresponding to the maximum length plₘₐₓ and a user-defined minimum length corresponding to the minimum length plₘᵢₙ, and the user-defined maximum length and the user-defined minimum length are smaller than the user-defined third length; plₛᵤₘ^{u} is used for representing a user-defined sum length corresponding to the sum length plₛᵤₘ; plₘₐₓ^{ur} is used for representing a user-defined maximum length ratio calculated by dividing the user-defined maximum length over the user-defined sum length; plₘᵢₙ^{ur} is used for representing a user-defined minimum length ratio calculated by dividing the user-defined minimum length over the user-defined sum length; and pl₃^{ur} is used for representing a user-defined third length ratio calculated by dividing the user-defined third length over the user-defined sum length.

For example, when calculating a maximum length difference productᵣ_{_{dir}}(plₘₐₓ) based on the above-mentioned formula of productᵣ_{_{dif}}(pl), it is firstly determine whether an absolute difference between the maximum length ratio plₘₐₓ^{r} and the user-defined maximum length ratio plₘₐₓ^{r} larger than the user-defined difference value t. In the situation that said absolute difference larger than the user-defined difference value t, the maximum length difference productᵣ_{_{dif}}(plₘₐₓ) is defined equal to said absolute difference. In the situation that said absolute difference not larger than the user-defined difference value t, the maximum length difference productᵣ_{_{dif}} (plₘₐₓ) is defined equal to zero. In the same manner, a minimum length difference productᵣ_{_{dif}} (plₘᵢₙ) and a third length difference productᵣ_{_{dif}}(pl₃) can be derived based on said formula of productᵣ_{_{dif}} (pl).

Particularly, in the ninth objective f₉, a user-defined maximum length ratio plₘₐₓ^{ur}, a user-defined minimum length ratio plₘᵢₙ^{ur}, a user-defined third length ration pl₃^{ur} and a user-defined difference value t can be determined by characteristics of a PCR target. In an instance, the user-defined difference value t is 5. However, it should be noted, the present invention is not limited to the numerical values mentioned above.

A tenth objective f₁₀ is provided. The tenth objective f₁₀ is adapted for calculating/evaluating an overall product length conformity level to reflect how many product lengths not less than a preset product length, and is defined by the following formula.

$${f}_{10} = {product}_{size} = \{ \begin{matrix}d 10 = 3 \\ if {pl}_{1} \geq 100 , d 10 - 1 \\ if {pl}_{2} \geq 100 , d 10 - 1 \\ if {pl}_{3} \geq 100 , d 10 - 1\end{matrix} ;$$

In the formula of the tenth objective f₁₀, product_{size} is used for presenting an indicator (lower is better) of an overall product length conformity level; d10 is an indicative value having a default value equal to 3 and is used for calculating the overall conformity level among product lengths; a total number of occurrences is initially defined to be zero, and in a condition that any to-be-evaluated product length is larger than or equal to 100 (referring to a base product length as said preset product length), the total number of occurrences is accumulated by 1; the tenth objective f₁₀/indicative value d10 is calculated by subtracting the total number of occurrences meeting said condition from the default value; said to-be-evaluated product lengths are respectively defined by the first length pl₁, the second length pl₂ and the third length pl₃.

Based on said first to tenth objectives f₁ to f₁₀, especially based on the second objective f₂, the fourth objective f₄, the sixth objective f₆, the seventh objective f₇ and the ninth objective f₉ which are distinct from the conventional common objectives, the designed results derived by the objectives proposed by this invention has better accuracy than that derived by the conventional common objectives under applying the same or similar assessment indicators/criteria. Said designed results are listed in the following TABLE 1. In TABLE 1, the “Algorithm A” represents NSGAII (Non-dominated Sorting Genetic Algorithm II) and using a corresponding model, and the “Algorithm B” represents MOPSO (Multiple Objective Particle Swarm Optimization) and using a corresponding model.

TABLE 1
The comparison on designed accuracy between objectives proposed
by this invention and the conventional common objectives.

| | Design Method | | | |
|---|---|---|---|---|
| | | | Inventive Objectives | |
| Assessment | Conventional Objectives | | (proposed by this invention) | |
| Indicators | Algorithm A | Algorithm B | Algorithm A | Algorithm B |
| GC % | 99.10% | 99.23% | 99.60% | 99.67% |
| GC_{clamp} | 68.76% | 69.15% | 71.68% | 72.10% |
| Tm | 64.42% | 67.49% | 91.95% | 92.82% |
| Tm_{dif} | 99.92%* | 99.92% | 97.51% | 98.13% |
| Dimerₙᵤₘ | 93.84% | 94.06% | 93.37% | 93.44% |
| Hairpinₙᵤₘ | 93.39% | 93.34% | 88.03% | 87.94% |
| Primer_{LEN} | 85.95% | 87.04% | 84.79% | 84.90% |
| Product_{LEN} | 84.15% | 91.69% | 99.99% | 100.00% |
| Specificity | 94.37% | 94.69% | 96.13% | 96.02% |

The definitions of the assessment indicators shown in TABLE 1 are elaborated as the following.
(1) “GC %” means a GC proportion of a designed primer and is qualified when the GC % ranges from 20% to 80%.
(2) “GC_{clamp}” can be also denoted/recognized as “GC clamp” and is qualified when the nucleotide type in 3′ end of a designed primer is G or C.
(3) “Tm” means a melting temperature of a designed primer and is qualified when the Tm ranges from 45° C. to 62° C.
(4) “Tm_{dif}” represents a level to assess a conformity/difference whether a melting temperature of a designed primer meets said Tm range (45° C. to 62° C.).
(5) “Dimerₙᵤₘ” means a total number of dimers generate between any two designed primers and is qualified when the Dimerₙᵤₘ is smaller than 5.
(6) “Hairpinₙᵤₘ” means a total number of hairpin structures formed in a designed primer and is qualified when the Hairpinₙᵤₘ is smaller than 5.
(7) “Primer_{LEN}” means a length of a designed primer and is qualified when the Primer_{LEN} ranges from 16 to 28.
(8) “Product_{LEN}” means a length of a PCR product formed by designed primers and is qualified when the Product_{LEN} ranges from 100 to 300.
(9) “Specificity” means a summation of each specificity of all designed primers and is qualified when the Specificity is smaller than 2.

From the results shown in TABLE 1, it can be observed, based on applying the algorithms A and B, the accuracies on some of the assessment indicators (such as the GC_{clamp} and Tm) of the primers designed by the conventional objectives are smaller than 70%; while the accuracies of all assessment indicators are greater than 70% by using the proposed objectives of this invention. More specifically, all the accuracies of the assessment indicator Tm obtained by the conventional objectives are smaller than 68%, while all the accuracies of the corresponding assessment indicator Tm obtained by the proposed objectives of this invention are higher than 91% so as to greatly improve the accuracy of the corresponding assessment indicator. Moreover, all the accuracies of the assessment indicator Product_{LEN} obtained by the conventional objectives are smaller than 92%, while all the accuracies of the corresponding assessment indicator (Product_{LEN}) obtained by the proposed objectives of this invention are higher than 99% so as to greatly improve the accuracy of the corresponding assessment indicator.

More specifically, said conventional objectives and the proposed objectives of this invention are the same in the first, third, fifth, eighth and tenth objectives, and are different in the second, fourth, sixth, seventh and ninth objectives. For those conventional objectives, different from the proposed objectives of this invention, are elaborated in the following.

The conventional second objective f₂′ is adapted for calculating/evaluating an overall GC proportion level reflecting an occurrence frequency that a corresponding GC proportion of each primer does not meet a preset GC proportion range, and is defined by the following formula.

$${{f}_{2}}^{′} = GC {{\%}_{range}}^{′} = \{ \begin{matrix}d {2}^{′} = 4 \\ if ( 20 \% \leq GC \% ( {fp}_{1} ) \leq 80 \% ) , d {2}^{′} - 1 \\ if ( 20 \% \leq GC \% ( {rp}_{1} ) \leq 80 \% ) , d {2}^{′} - 1 \\ if ( 20 \% \leq GC \% ( {fp}_{2} ) \leq 80 \% ) , d {2}^{′} - 1 \\ if ( 20 \% \leq GC \% ( {rp}_{2} ) \leq 80 \% ) , d {2}^{′} - 1\end{matrix}$$

In the formula of the conventional second objective f₂′, d2′ is an indicative value having a default value equal to 4 and is used for calculating the overall GC proportion level; a total number of occurrences is initially defined to be zero, and in a condition that any to-be-evaluated GC proportion is not smaller than 20% and not greater than 80%, the total number of occurrences is accumulated by 1; the conventional second objective f₂′/the indicative value d2′ is calculated by subtracting a total number of occurrences meeting said condition from the default value; said to-be-evaluated GC proportions are respectively defined by a corresponding GC proportion of a respective one of the first forward primer fp₁, the first reverse primer rp₁, the second forward primer fp₂ and the second reverse primer rp₂.

The conventional fourth objective f₄′ is adapted for calculating/evaluating an overall melting temperature level reflecting an occurrence frequency that a corresponding melting temperature of each primer does not meet a preset melting temperature range, and is defined by the following formula.

$${{f}_{4}}^{′} = {{Tm}_{range}}^{′} = \{ \begin{matrix}d {4}^{′} = 4 \\ if ( 45 ° C . \leq {Tm}_{BM} ( {fp}_{1} ) \leq 62 ° C . ) , d {4}^{′} - 1 \\ if ( 45 ° C . \leq {Tm}_{BM} ( {rp}_{1} ) \leq 62 ° C . ) , d {4}^{′} - 1 \\ if ( 45 ° C . \leq {Tm}_{BM} ( {fp}_{2} ) \leq 62 ° C . ) , d {4}^{′} - 1 \\ if ( 45 ° C . \leq {Tm}_{BM} ( {rp}_{2} ) \leq 62 ° C . ) , d {4}^{′} - 1\end{matrix} ;$$

In the formula of the conventional fourth objective f₄′, d4′ is an indicative value having a default value equal to 4 and is used for calculating the overall melting temperature level; a total number of occurrences is initially defined to be zero, and in a condition that any to-be-evaluated melting temperature is not smaller than 45° C. and not greater than 62° C., the total number of occurrences is accumulated by 1; the conventional fourth objective f₄′/the indicative value d4′ is calculated by subtracting a total number of occurrences meeting said condition from the default value; said to-be-evaluated melting temperatures are respectively defined by a corresponding melting temperature of a respective one of the first forward primer fp₁, the first reverse primer rp₁, the second forward primer fp₂ and the second reverse primer rp₂, and are calculated based on the Bolton and McCarthy formula.

The conventional sixth objective f₆′ is adapted for calculating/evaluating an overall dimer formation level reflecting an occurrence frequency that a dimer is formed between any two primers, and is defined by the following formula.

$${{f}_{6}}^{′} = {Dimer}^{′} = \{ \begin{matrix}d {6}^{′} = 10 \\ if {fp}_{1} , {rp}_{1} do not form a {dimer}_{cross} , d {6}^{′} - 1 \\ if {fp}_{1} , {rp}_{2} do not form a {dimer}_{cross} , d {6}^{′} - 1 \\ if {fp}_{2} , {rp}_{1} do not form a {dimer}_{cross} , d {6}^{′} - 1 \\ if {fp}_{2} , {rp}_{2} do not form a {dimer}_{cross} , d {6}^{′} - 1 \\ if {fp}_{1} , {fp}_{2} do not form a {dimer}_{cross} , d {6}^{′} - 1 \\ if {rp}_{1} , {rp}_{2} do not form a {dimer}_{cross} , d {6}^{′} - 1 \\ if {fp}_{1} , {fp}_{1} do not form a {dimer}_{self} , d {6}^{′} - 1 \\ if {rp}_{1} , {rp}_{1} do not form a {dimer}_{self} , d {6}^{′} - 1 \\ if {fp}_{2} , {fp}_{2} do not form a {dimer}_{self} , d {6}^{′} - 1 \\ if {rp}_{2} , {rp}_{2} do not form a {dimer}_{self} , d {6}^{′} - 1\end{matrix} ;$$

In the formula of the conventional sixth objective f₆′, d6′ is an indicative value having a default value equal to 10 and is used for calculating the overall dimer formation level; a total number of occurrences is initially defined to be zero, and in a condition that any combination of two identical or distinct primers forms a dimer, the total number of occurrences is accumulated by 1; the conventional sixth objective f₆′/the indicative value d6′ is calculated by subtracting a total number of occurrences meeting said condition from the default value; dimer_{cross} means a dimer formed by two distinct primers; dimer_{self} means a dimer formed by two identical primers.

The conventional seventh objective f₇′ is adapted for calculating/evaluating an overall hairpin formation level reflecting an occurrence frequency that at least one hairpin structure is formed in each primer, and is defined by the following formula.

$${{f}_{7}}^{′} = {Hairpin}^{′} = \{ \begin{matrix}d {7}^{′} = 4 \\ if {fp}_{1} does not form a Hairpin , d {7}^{′} - 1 \\ if {fp}_{1} does not form a Hairpin , d {7}^{′} - 1 \\ if {fp}_{1} does not form a Hairpin , d {7}^{′} - 1 \\ if {fp}_{1} does not form a Hairpin , d {7}^{′} - 1\end{matrix} ;$$

In the formula of the conventional seventh objective f₇′, d7′ is an indicative value having a default value equal to 4 and is used for calculating the overall hairpin formation level; a total number of occurrences is initially defined to be zero, and in a condition that any primer has at least one hairpin structure, the total number of occurrences is accumulated by 1; the conventional seventh objective f₇′/the indicative value d7′ is calculated by subtracting a total number of occurrences meeting said condition from the default value.

The conventional ninth objective f₉′ is adapted for calculating/evaluating an overall difference of product lengths formed by different primer pairs, and is defined by the following formula.

$${{f}_{9}}^{′} = {{product}_{ratio}}^{′} = 5 - ( {ratio}_{max} - {ratio}_{min} ) ;$$

$${{pl}_{sum}}^{′} = {pl}_{max} + {pl}_{min} ;$$

$${pl}_{max} : {pl}_{min} : {{pl}_{sum}}^{′} = {ratio}_{max} : {ratio}_{min} : 5 ;$$

In the conventional ninth objective f₉′, productᵣₐₜᵢₒ′ is used for representing an indicator (lower is better) of overall difference of product lengths formed by different primer pairs; plₛᵤₘ′ is used for representing the sum length by summing the maximum length plₘₐₓ and the minimum length plₘᵢₙ; by making the ratio of the sum length plₛᵤₘ′ to be 5 (as an ratioₛᵤₘ′), ratioₘₐₓ is defined by multiplying the maximum length plₘₐₓ with 5 (ratioₛᵤₘ′) and then dividing by the sum length plₛᵤₘ′, and ratioₘᵢₙ is defined by multiplying the minimum length plₘᵢₙ with 5 (ratioₛᵤₘ′) and then dividing by the sum length plₛᵤₘ′. Consequently, the indicator of the productᵣₐₜᵢₒ′ can be calculated. In the design and calculation for primer pairs by applying above-mentioned conventional objectives (optimized by said Algorithms A and B), the population size is set to 50, and the maximum number of iterations is set to 1500. In the conventional second objective f₂′, the GC proportion is set between 20% to 80% (a lower boundary value is 20% and a higher boundary value is 80%). In the conventional fourth objective f₄′, the melting temperature is set between 45° C.˜62° C. (a lower boundary value is 45° C. and a higher boundary value 62° C.).

In the design and calculation for primer pairs by applying the proposed objectives of this invention (optimized by said Algorithms A and B), the population size is set to 50, and the maximum number of iterations is set to 100. In the second objective f₂ of this invention, the maximum value of the preset GC proportion GC % max and the minimum value of the preset GC proportion GC % min are both set to 50%. In the fourth objective f₄ of this invention, the maximum value of the preset melting temperature Tmₘₐₓ and the minimum value Tmₘᵢₙ of the preset melting temperature are both set to 50° C. In the sixth objective f₆ of this invention, the user-defined primer biding quantity Dimerᵤₛₑᵣ is set to zero. In the seventh objective f₇ of this invention, the user-defined primer hairpin structure quantity Hairpinᵤₛₑᵣ is set to zero. In the ninth objective f₉, the user-defined maximum length ratio plₘₐₓ^{ur} is set between 28 and 38 (preferably set to 33), the user-defined max minimum length ratio plₘᵢₙ^{ur} is set between 15 and 25 (preferably set to 20), and the user-defined third length ratio pl₃^{ur} is set between 42 and 52 (preferably set to 47).

Based on the above-mentioned objectives of this invention, the conventional objectives and TABLE 1, it can be understood that the differences in accuracy are originated from the essential differences among the objectives among this invention and the conventional objectives. Especially, by comparing the difference objectives as the second, fourth, sixth, seventh and ninth objectives, it can be clearly understood that those mentioned conventional objectives focus more on “the existence” of interested characteristics to determine whether or not meet corresponding conditions, while the objectives of this invention can not only reflect “the existence” of the interested characteristics but also render “the detailed magnitudes/degrees” of the interested characteristics. In other words, in said objectives of this invention, when an interested characteristic meets or exceeds (preferably when exceeds) a preset condition, the corresponding indicator can render the difference magnitude to the preset condition. Therefore, the objectives of this invention contribute on providing detailed difference magnitudes to corresponding preset conditions to make corresponding indicators have better discriminability for designing better primer pairs.

In a preferred embodiment, as shown in TABLE 2, by applying said objectives of this invention with a specific Algorithm C, a design result having the best performance/accuracy is obtained. Said Algorithm C, representing GPAWOA (Guided Population Archive Whale Optimization Algorithm), is cited from the journal contents as recited in “SUMMARY OF THE INVENTION” of the specification of this invention; that is, the following mentioned contents and steps of said Algorithm C can be understood by a person having ordinary skill in the art of this invention.

TABLE 2
The comparison on designed accuracy among
the Algorithm C and other Algorithms A, B.

| | Design Method | | |
|---|---|---|---|
| | Conventional | Inventive Objectives | |
| Assessment | Objectives | (proposed by this invention) | |
| Indicators | Algorithm A or B | Algorithm A or B | Algorithm C |
| GC % | 99.23% (B) | 99.67% (B) | 99.98% |
| GC_{clamp} | 69.15% (B) | 72.10% (B) | 77.98% |
| Tm | 67.49% (B) | 92.82% (B) | 97.23% |
| Tm_{dif} | 99.92% (A \grave{ } B) | 98.13% (B) | 99.96% |
| Dimerₙᵤₘ | 94.06% (B) | 93.44% (B) | 98.61% |
| Hairpinₙᵤₘ | 93.39% (A) | 88.03% (A) | 95.43% |
| Primer_{LEN} | 87.04% (B) | 84.90% (B) | 89.00% |
| Product_{LEN} | 91.69% (B) | 100.00% (B) | 100.00% |
| Specificity | 94.69% (B) | 96.13% (A) | 95.94% |

In TABLE 2, for the columns denoted as “Algorithm A or B”, each of the corresponding accuracy is shown by a better result with a corresponding Algorithm A or B, and the letter “A” or “B” in a parentheses indicates the corresponding Algorithm A or B as the calculation basis for said better results. For example, for the assessment indicator as GC %, by applying the conventional objectives, the accuracy has highest value 99.23% derived from the Algorithm B; by applying the objective of this invention, the accuracy has highest value 99.67% also derived from the Algorithm B. In another example, for the assessment indicator as Specificity, by applying the conventional objectives, the accuracy has highest value 94.69% derived from the Algorithm B; by applying the objective of this invention, the accuracy has highest value 96.13% derived from the Algorithm A.

Additionally, in TABLE 2, to emphasize the best designed results by applying the objectives of this invention with the Algorithm C, said best results, derived by the Algorithm C, are independently shown in the corresponding table column. The accuracy of GC_{clamp} is 77.98%, the accuracy of Primer_{LEN} is 89.00%, and the accuracies on other assessment indicators all are higher than 95%. Especially, eight of the nine assessment indicators (except the Specificity) respectively have the best accuracy. Therefore, based on the objectives of this invention, TABLE 2 can prove that comparing with the Algorithms A and B, the overall design result derived from the Algorithm C has better performance (especially significantly improve the accuracies of Tm, Dimerₙᵤₘ, Hairpinₙᵤₘ and Primer_{LEN}).

Especially, based on a further research of this invention, it is proposed that comparing to the conventional objectives, the corresponding design results obtained by applying specific combinations of several specific objectives selected from the complete objectives of this invention for designing the primer pairs (referring as the confronting two-pair primers) also have better performance/accuracy. Said specific combinations of several specific objectives are presented in the following TABLE 3, and the corresponding design results are shown in the following TABLE 4.

TABLE 3
Specific combinations with corresponding objectives
selected from the objectives of this invention.

| | Selected Objectives | Design Result |
|---|---|---|
| Complete | Objectives f₁-f₁₀ | See TABLEs 2 and 4 |
| Combination | | |
| First | Objectives f₄ and f₉ | See TABLE 4 |
| Combination | | |
| Second | Objectives f₄, f₈ and f₉ | See TABLE 4 |
| Combination | | |
| Third | Objectives f₄, f₆, f₇ and f₉ | See TABLE 4 |
| Combination | | |
| Fourth | Objectives f₄, f₆, f₇, f₈ and f₉ | See TABLE 4 |
| Combination | | |

TABLE 4
The design results obtained by using said specific
combination of objectives and the Algorithm C.

| | Design Method | | | | | |
|---|---|---|---|---|---|---|
| | Conventional | First | Second | Third | Fourth | Complete |
| | Objectives | Comb. | Comb. | Comb. | Comb. | Comb. |
| Assess. | Algorithm | (f₄, f₉) | (f₄, f₈, f₉) | (f₄, f₆, f₇, f₉) | (f₄, f₆-f₉) | (f₁-f₁₀) |
| Indicators | A or B | Algorithm C | | | | |
| GC % | 99.23% | 99.91% | 99.93% | 99.98% | 99.96% | 99.98% |
| GC_{clamp} | 69.15% | 72.83% | 73.57% | 72.91% | 73.31% | 77.98% |
| Tm | 67.49% | 99.19% | 99.25% | 82.39% | 81.76% | 97.23% |
| Tm_{dif} | 99.92% | 99.78% | 99.81% | 99.73% | 99.72% | 99.96% |
| Dimerₙᵤₘ | 94.06% | 96.30% | 96.38% | 98.93% | 98.88% | 98.61% |
| Hairpinₙᵤₘ | 93.39% | 94.43% | 95.43% | 99.50% | 99.41% | 95.43% |
| Primer_{LEN} | 87.04% | 87.08% | 87.15% | 87.21% | 87.09% | 89.00% |
| Product_{LEN} | 91.69% | 100.00% | 100.00% | 100.00% | 100.00% | 100.00% |
| Specificity | 94.69% | 96.75% | 97.75% | 97.00% | 97.60% | 95.94% |

| | | | | | |
|---|---|---|---|---|---|
| Calculation Time (ms) | 31.30 | 1787.41 | 1760.38 | 2691.28 | 3845.69 |

From TABLE 4, the corresponding design results can be understood as the following.
(1) In the first combination (corresponding to the fourth and ninth objectives of this invention), except for the assessment indicator Tm_{dif} just slightly lower by 0.14% compared to the conventional result, the accuracies of all the other eight assessment indicators are superior to the conventional result (derived by using conventional objectives and the Algorithm A and/or B). Particularly, to generated/calculated the design result, the corresponding the calculation time requires only 31.30 ms and is at least 56 times faster than any calculation time of the other combinations. That is, in the consideration to get a design result with a minimum/fastest calculation time, the first combination can be the optimal design objectives.
(2) In the second combination (corresponding to the fourth, eighth and ninth objectives of this invention), except for the assessment indicator Tm_{dif} just slightly lower by 0.11% compared to the conventional result, the accuracies of all the other eight assessment indicators are superior to the conventional result. Besides, the accuracies of all nine assessment indicators are superior to the first combination. The calculation time is 1787.41 ms. Further, the corresponding assessment indicators Tm (99.25%) and Specificity (97.75%) are the optimal results over the other combinations.
(3) In the third combination (corresponding to the fourth, sixth, seventh and ninth objectives of this invention), except for the assessment indicator Tm_{dif} just slightly lower by 0.18% compared to the conventional result, the accuracies of all the other eight assessment indicators are superior to the conventional result. The calculation time is 1760.38 ms. Further, the corresponding assessment indicators GC % (99.98%), Dimerₙᵤₘ(98.93%) and Hairpinₙᵤₘ(99.50%) are the optimal results over the other combinations. Moreover, compared to the first combination, the corresponding assessment indicators GC %, GC_{clamp}, Dimerₙᵤₘ, Hairpinₙᵤₘ, Primer_{LEN} and Specificity are better. Compared to the second combination, the third combination has better performance in the assessment indicators GC %, Dimerₙᵤₘ, Hairpinₙᵤₘ and Primer_{LEN}, while the second combination has better performance in the assessment indicators GC_{clamp}, Tm, Tm_{dif} and Specificity.
(4) In the fourth combination (corresponding to the fourth, sixth, seventh, eighth and ninth objectives of this invention) except for the assessment indicator Tm_{dif} just slightly lower by 0.20% compared to the conventional result, the accuracies of all the other eight assessment indicators are superior to the conventional result. The calculation time is 2691.28 ms. Compared to the first combination, the fourth combination has better performance in the assessment indicators GC %, GC_{clamp}, Dimerₙᵤₘ, Hairpinₙᵤₘ, Primer_{LEN} and Specificity. Compared to the second combination, the fourth combination has better performance in the assessment indicators GC %, Dimerₙᵤₘ and Hairpinₙᵤₘ. Compared to the third combination, the fourth combination has better performance in the assessment indicators GC_{clamp} and Specificity.

(5) In the complete combination (corresponding to the first to tenth objectives of this invention), all of the nine assessment indicators are superior to the conventional result. The corresponding assessment indicators GC % (99.98%), GCcₗₐₘₚ (77.98%), Tm_{dif}(99.96%) and Primer_{LEN} (89.00%) are the optimal results over the other combinations. Compared to the first combination, the complete combination has better performance in the assessment indicators GC %, GC_{clamp}, Tm_{dif}, Dimerₙᵤₘ, Hairpinₙᵤₘ and Primer_{LEN} are better. Compared to the second combination, the complete combination has better performance in the assessment indicators GC %, GC_{clamp}, Tm_{dif}, Dimerₙᵤₘ and Primer_{LEN}. Compared to the third combination, the complete combination has better performance in the assessment indicators GC_{clamp}, Tm, Tm_{dif}, and Primer_{LEN}. Compared to the fourth combination, the complete combination has better performance in the assessment indicators GC %, GC_{clamp}, Tm, Tm_{dif}, and Primer_{LEN}. That is, this complete combination overall has the optimal results over the other combinations; nevertheless, this complete combination has the longest calculation time (3845.69 ms).

Based on the above-mentioned design objectives proposed by this invention and the corresponding calculation means (as the Algorithm A, B or C), this invention provides a multi-objective design method for confronting two-pair primers. Said method includes an inputting step S1 and a calculating step S2, and is implemented by a computer including at least a processor and a non-transitory memory.

In the inputting step S1, a DNA template fragment and a corresponding design objective are provided/inputted. Specifically, said DNA template fragment and said corresponding design objective are provided/inputted in the computer. Referring to the sole FIGURE, said DNA template fragment includes a forward chain information and a reverse chain information. The forward chain information includes compositions of a forward chain FC and a nucleotide variant site NVS located on the forward chain FC. The reverse chain information includes compositions of a reverse chain RC and a nucleotide variant site NVS located on the reverse chain RC.

As the contents shown in TABLE 3, said design objective may include at least said fourth objective f₄ and said ninth objective f₉, in this condition, said design objective includes the above-mentioned first combination of the specific objectives (f₄ and f₉). Optionally, besides said first combination of objectives, said design objective may additionally include said eighth objective f₈; in this condition, said design objective includes the above-mentioned second combination of the specific objectives (f₄, f₈ and f₉). Optionally, besides said first combination of objectives, said design objective may additionally include said sixth objective f₆ and said seventh objective f₇; in this condition, said design objective includes the above-mentioned third combination of the specific objectives (f₄, f₆, f₇ and f₉). Optionally, besides said first combination of objectives, said design objective may additionally include said sixth objective f₆, said seventh objective f₇ and said eighth objective f₈; in this condition, said design objective includes the above-mentioned fourth combination of the specific objectives (f₄, f₆, f₇, f₈ and f₉). Preferably, said design objective includes said first to tenth objectives f₁-f₁₀; in this condition, said design objective corresponds to the above-mentioned complete combination of the specific objectives (f₁-f₁₀).

In the calculating step S2, according to said DNA template segment and said design objective, a corresponding calculation means is applied to assess/evaluate a score/value of at least one set of confronting two-pair primers corresponding to the design objective so as to determine at least one set of confronting two-pair primers to be an optimal solution. Specifically, the computer includes a corresponding pre-established calculation model, which may store in said non-transitory memory. Said pre-established calculation model is adapted for executing the calculation means to generate at least one to-be-assessed set of confronting two-pair primers, to generate corresponding score(s) (based on the design objective) for the at least one to-be-assessed set of confronting two-pair primers, and to determine whether the at least one to-be-assessed set of confronting two-pair primers can be the optimal solution suitable for applying in the process of PCR-CTPP. Said calculation means may, but not limited to, be one of the above-mentioned Algorithms A, B and C. Preferably, as shown in TABLEs 2 and 4, primers of at least one set of confronting two-pair primers generated by the Algorithm C with the specific objectives (as the combinations shown in TABLE 3) have better PCR efficiency and accuracy.

Referring to the sole FIGURE, said one set of confronting two-pair primers includes a first forward primer fp₁, a first reverse primer rp₁, a second forward primer fp₂ and a second reverse primer rp₂. The first forward primer fp₁ has a first forward primer length fl₁. The first reverse primer rp₁ has a first reverse primer length rl₁, and has a nucleotide variant site NVS corresponding to the nucleotide variant site NVS of the forward chain FC. The second forward primer fp₂ has a second forward primer length fl₂, and has a nucleotide variant site NVS corresponding to the nucleotide variant site NVS of the reverse chain RC. The second reverse primer rp₂ has a second reverse primer length rl₂. The first forward primer fp₁ and the first reverse primer rp₁ are defined to be the first primer pair having a first length pl₁. The second forward primer fp₂ and the second reverse primer rp₂ are defined to be the second primer pair having a second length pl₂. The first forward primer fp₁ and the second reverse primer rp₂ define a third length pl₃.

It shall be noted, by using the calculation method to assess scores (corresponding to values calculated by corresponding objectives of the design objective) of the at least one set of confronting two-pair primers, the so-called optimal solution is determined by at least one set of confronting two-pair primers overall having minimum scores in every objectives of the design objective. Preferably, the optimal solution is obtained by applying a Pareto Chart Analysis in said calculation means (especially one of the Algorithms A-C). By applying the Pareto Chart Analysis, in a condition that the optimal solution is a single set of the confronting two-pair primers, each of the scores of the objectives of the single set of the confronting two-pair primers of the optimal solution dominates a respective one of the scores of the objectives of any other sets of the confronting two-pair primers. Also, by applying the Pareto Chart Analysis, in the condition that the optimal solution is multiple sets of the confronting two-pair primers, the scores of the objectives of the multiple sets of the confronting two-pair primers of the optimal solution dominate each other, and each of the scores of the objectives of the multiple sets of the confronting two-pair primers of the optimal solution dominates a respective one of the scores of the objectives of any other sets of the confronting two-pair primers. It should be noted, said definition of “dominate” in the Pareto Chart Analysis can be understood by a skilled person in the art, and corresponding contents can, but not limited to, be found by referring to the overall contents disclosed in U.S. patent publication No. 11862296 B2 (where “Pareto Chart tool” recited in the mentioned patent corresponds to “Pareto Chart Analysis” of this invention).

Specifically, said Algorithm C is the Guided Population Archive Whale Optimization Algorithm, and includes the following main steps: a calculating step, a filtering step, a termination checking step, a position updating step and a boundary assessing step. In the calculating step, the scores of at least one set of the confronting two-pair primers are calculated/obtained by the objectives of the design objective. In the filtering step, based on said Pareto Chart Analysis, the at least one set of the confronting two-pair primers having the scores analyzed to be the optimal solution is filtered in a pareto set. A respective one crowding distance for each score in a pareto set is calculated to determine a candidate solution with a minimum or maximum value of the crowding distances. In the termination checking step, a current accumulated number of a computing iteration is checked whether meeting a preset termination number of the computing iteration or not. If the current accumulated number of the computing iteration meets (equals) the preset termination number of the computing iteration, a set of the confronting two-pair primers corresponding to a current candidate solution is defined to be an optimal set of the confronting two-pair primers for corresponding PCR-CTPP process. If the current accumulated number of the computing iteration does not meet (is larger than) the preset termination number of the computing iteration, the following steps are executed continuously. In the position updating step, based on the candidate solution to update a position of a whale group so as to generate at least one updated sets of the confronting two-pair primers. In the boundary assessing step, each length of primers in an updated set of the confronting two-pair primers is checked whether meeting a preset range of length or not. If each length of primers meets (does not exceed) a preset range of length, the updated set of the confronting two-pair primers are inputted into the calculation step to proceed a new run of the computing iteration (accumulated number of the computing iteration is updated accordingly). If any length of primers does not meet (exceeds) a preset range of length, new sets of the confronting two-pair primers, in which each length of primers meets the preset range of length, are generated based on a preset rule and then are inputted into the calculation step to proceed a new run of the computing iteration.

In summary, in the multi-objective design method for confronting two-pair primers, by applying the proposed design objective adapted for reflecting the approximation/deviation extents to various interested characteristics or different preset conditions, the design results obtained by various calculation means overall have better performance than that obtained by conventional design objective. In this respect, the designed set of confronting two-pair primers, derived by said designed results (corresponding to said optimal solution or candidate solution), has improved accuracies to meet the requirements of the assessment indicators, and the corresponding PCR efficiency and accuracy are also improved accordingly. Further, by applying said proposed design objective with specific calculation means (corresponding to the Algorithm C which is the Guided Population Archive Whale Optimization Algorithm), the performance of the designed result can be further improved (comparing to other calculation means such as Algorithms A and B), and hence the corresponding PCR efficiency and accuracy are improved.

Although the present invention has been described with respect to the above preferred embodiments, these embodiments are not intended to restrict the present invention. Various changes and modifications on the above embodiments made by any person skilled in the art without departing from the spirit and scope of the present invention are still within the technical category protected by the present invention. Accordingly, the scope of the present invention shall include the literal meaning set forth in the appended claims and all changes which come within the range of equivalency of the claims.

What is claimed is:

1. A multi-objective design method for confronting two-pair primers, executed by a computer to execute following steps, comprising:
inputting a DNA template fragment and a design objective; and
applying a calculation means to assess scores of at least one set of confronting two-pair primers according to the DNA template and the design objective so as to determine an optimal solution from the at least one set of confronting two-pair primers;
wherein, the DNA template fragment includes a forward chain information and a reverse chain information; the forward chain information includes compositions of a forward chain and a nucleotide variant site located on the forward chain; and the reverse chain information includes compositions of a reverse chain and a nucleotide variant site located on the reverse chain;
wherein, said one set of confronting two-pair primers includes a first forward primer, a first reverse primer, a second forward primer and a second reverse primer; the first forward primer has a first forward primer length; the first reverse primer has a first reverse primer length, and has a nucleotide variant site corresponding to the nucleotide variant site of the forward chain; the second forward primer has a second forward primer length, and has a nucleotide variant site corresponding to the nucleotide variant site of the reverse chain; the second reverse primer has a second reverse primer length; the first forward primer and the first reverse primer are defined to be a first primer pair having a first length; the second forward primer and the second reverse primer are defined to be a second primer pair having a second length; and the first forward primer and the second reverse primer define a third length;
wherein, the design objective includes a fourth objective and a ninth objective;
wherein, the fourth objective is adapted for calculating an overall difference level by summing each differences level between a melting temperature of one of the primers and a preset melting temperature range, and is defined by the following formula:
$${f}_{4} = {Tm}_{dif} ( {fp}_{1} ) + {Tm}_{dif} ( {rp}_{1} ) + {Tm}_{dif} ( {fp}_{2} ) + {Tm}_{dif} ( {rp}_{2} ) ;$$
$${Tm}_{BM} ( primer ) = \{ \begin{matrix}{Tm}_{min} - {Tm}_{BM} ( primer ) , if {Tm}_{BM} ( primer ) < {Tm}_{min} \\ {Tm}_{BM} ( primer ) - {Tm}_{max} , if {Tm}_{BM} ( primer ) > {Tm}_{max} \\ 0 , if {Tm}_{min} \leq {Tm}_{BM} ( primer ) \leq {Tm}_{max}\end{matrix} ;$$
$${Tm}_{BM} ( primer ) = 81.5 + 166 \times ( log [ {Na}^{+} ] ) + 0.41 \times GC \% - 675 / {primer}_{LEN} ;$$
wherein, f₄ is used for the fourth objective; Tm_{dif}(primer) is used for representing a difference level between a melting temperature of one of the primers and the preset melting temperature range; Tm_{BM}(primer) is used for representing the melting temperature for each one of the primers, and is calculated based on a Bolton and McCarthy formula; [Na⁺] is used for representing a molarity of Na ions of a solvent where a respective one primer is added; primer_{LEN} is used for representing a length of the respective one primer; Tmₘₐₓ and Tmₘᵢₙ are respectively used for representing a maximum value and a minimum value of the preset melting temperature range so as to mutually define a value range of the preset melting temperature range; fp₁, rp₁, fp₂ and rp₂ are respectively used for representing a first forward primer, a first reverse primer, a second forward primer and a second reverse primer in a corresponding one set of confronting two-pair primers; Tm_{dif}(fp₁), Tm_{dif}(rp₁), Tm_{dif}(fp₂) and Tm_{dif}(rp₂) are respectively used for representing the Tm_{dif}(primer) of the corresponding one of the first forward primer, the first reverse primer, the second forward primer and the second reverse primer; and
wherein, the ninth objective is adapted for calculating a product length difference level by summing each difference between a product length formed by one of primer pairs and a user-defined product length formed by a corresponding one of the primer pairs, and is defined by the following formula:
$${f}_{9} = {product}_{{r}_{dif}} ( {pl}_{max} ) + {product}_{{r}_{dif}} ( {pl}_{min} ) + {product}_{{r}_{dif}} ( {pl}_{3} ) ;$$
$${product}_{{r}_{dif}} ( pl ) = \{ \begin{matrix}\vert {pl}^{r} - {pl}^{ur} \vert , & \vert {pl}^{r} - {pl}^{ur} \vert > t \\ 0 , & \vert {pl}^{r} - {pl}^{ur} \vert \leq t\end{matrix} ;$$
$$\{ \begin{matrix}{pl}_{sum} = {pl}_{max} + {pl}_{min} + {pl}_{3} \\ {pl}_{max}^{r} = {pl}_{max} \times \frac{100}{{pl}_{sum}} \\ {pl}_{min}^{r} = {pl}_{min} \times \frac{100}{{pl}_{sum}} \\ {pl}_{3}^{r} = {pl}_{3} \times \frac{100}{{pl}_{sum}}\end{matrix} and \{ \begin{matrix}{pl}_{sum}^{u} = {pl}_{max}^{u} + {pl}_{min}^{u} + {pl}_{3}^{u} \\ {pl}_{max}^{u} = {pl}_{max}^{u} \times \frac{100}{{pl}_{sum}^{u}} \\ {pl}_{min}^{u} = {pl}_{min}^{u} \times \frac{100}{{pl}_{sum}^{u}} \\ {pl}_{3}^{u} = {pl}_{3}^{u} \times \frac{100}{{pl}_{sum}^{u}}\end{matrix} ;$$
wherein, f₉ is used for the ninth objective; productᵣ_{_{dif}}(pl) is used for representing a definition on calculating a difference value for a ratio of the product length of a primer pair and a ratio of a user-defined product length; t is used for representing a user-defined difference value defining a predetermined difference ratio of the product length; pl^{r} is used for representing a ratio of the product length of one of the primer pairs; pl^{ur} is used for representing a ratio of the user-defined product length of a primer pair corresponding to pl^{r}; plₘₐₓ is used for representing a greater length between the first length and the second length, and is defined to be a maximum length; plₘᵢₙ is used for representing a smaller length between the first length and the second length, and is defined to be a minimum length; pl₃ is used for representing the third length which is larger than the maximum length and the minimum length; plₛᵤₘ is used for representing a sum length calculated by summing the first length, the second length and the third length; plₘₐₓ^{r} is used for representing a maximum length ratio calculated by dividing the maximum length over the sum length; plₘᵢₙ^{r} is used for representing a minimum length ratio calculated by dividing the minimum length over the sum length; pl₃^{r} is used for representing a third length ratio calculated by dividing the third length over the sum length; ply is used for representing a user-defined third length corresponding to the third length; plₘₐₓ^{u} and plₘᵢₙ^{u} are respectively used for representing a user-defined maximum length corresponding to the maximum length plₘₐₓ and a user-defined minimum length corresponding to the minimum length plₘᵢₙ, and both the user-defined maximum length and the user-defined minimum length are smaller than the user-defined third length; plₛᵤₘ^{u} is used for representing a user-defined sum length corresponding to the sum length plₛᵤₘ; plₘₐₓ^{ur} is used for representing a user-defined maximum length ratio calculated by dividing the user-defined maximum length over the user-defined sum length; plₘᵢₙ^{ur} is used for representing a user-defined minimum length ratio calculated by dividing the user-defined minimum length over the user-defined sum length; and pl₃^{ur} is used for representing a user-defined third length ratio calculated by dividing the user-defined third length over the user-defined sum length.

2. The multi-objective design method for confronting two-pair primers as claimed in claim 1, wherein the design objective further includes at least one of a first objective, a second objective, a third objective, a fifth objective, a sixth objective, a seventh objective, an eighth objective and a tenth objective; the first objective is used for calculating a difference level among the lengths of the primers; the second objective is used for calculating an overall difference level by summing each differences level between a proportion of nucleotides G and C in one of the primers and a preset GC proportion range; the third objective is used for calculating an overall stability of the primers; the fifth objective is used for calculating an overall difference level by summing each difference between the melting temperature of one of the primers and an average melting temperature of the primers; the sixth objective is used for calculating a dimer formation level for self-dimerization and cross-dimerization among the primers; the seventh objective is used for calculating an overall hairpin structure formation level of the primers; the eighth objective is used for calculating an overall specificity of the primers; and the tenth objective is used for calculating an overall product length conformity level to reflect how many product lengths not less than a preset product length.

3. The multi-objective design method for confronting two-pair primers as claimed in claim 2, wherein the first objective is defined by the following formula:
$${f}_{1} = \{ \begin{matrix}d 1 = 3 \\ if ( \vert {fl}_{1} - {rl}_{1} \vert \leq 3 ) , d 1 - 1 \\ if ( \vert {fl}_{2} - {rl}_{2} \vert \leq 3 ) , d 1 - 1 \\ if ( \vert {fl}_{1} - {rl}_{2} \vert \leq 3 ) , d 1 - 1\end{matrix} ;$$
wherein, f₁ is used for representing the first objective; fl₁, fl₂, rl₁ and rl₂ are respectively used for representing the first forward primer length, the second forward primer length, the first reverse primer length and the second reverse primer length; d1 is an indicative value having a default value equal to 3 and is used for calculating the overall difference level among lengths of the primers; a total number of occurrences is initially defined to be zero, and in a condition that any to-be-evaluated length difference is not larger than 3, the total number of occurrences is accumulated by 1; the first objective is calculated by subtracting the total number of occurrences meeting said condition from the default value; said to-be-evaluated length differences are respectively defined by an absolute value calculated by deducting the first reverse primer length from the first forward primer length, an absolute value calculated by deducting the second reverse primer length from the second forward primer length, and an absolute value calculated by deducting the second reverse primer length from the first forward primer length;
wherein, the second objective is defined by the following formula:
$${f}_{2} = GC {\%}_{range} = GC {\%}_{dif} ( {fp}_{1} ) + GC {\%}_{dif} ( {rp}_{1} ) + GC {\%}_{dif} ( {fp}_{2} ) + GC {\%}_{dif} ( {rp}_{2} ) ;$$
$$GC {\%}_{dif} ( primer ) = \{ \begin{matrix}GC {\%}_{min} - GC \% ( primer ) , if GC \% ( primer ) < GC {\%}_{min} \\ GC \% ( primer ) - GC {\%}_{max} , if GC \% ( primer ) > GC {\%}_{max} \\ 0 , if GC {\%}_{min} \leq GC \% ( primer ) \leq GC {\%}_{max}\end{matrix} ;$$
wherein, f₂ is used for representing the second objective; GC %_{dif}(primer) is used for representing a difference level between a GC proportion in one of the primers and the preset GC proportion range; GC % (primer) is used for representing a GC proportion in one of the primers; GC %ₘₐₓ and GC %ₘᵢₙ are respectively used for representing a maximum value and a minimum value of the preset GC proportion range so as to mutually define a value range of the preset GC proportion range; GC %_{dif}(fp₁), GC %_{dif}(rp₁), GC %_{dif}(fp₂) and GC %_{dif}(rp₂) are respectively used for representing the GC %_{dif}(primer) of the first forward primer, the first reverse primer, the second forward primer and the second reverse primer;
wherein, the third objective is defined by the following formula:
$${f}_{3} = \{ \begin{matrix}d 3 = 4 \\ if ( {3}^{′} of {fp}_{1} is G or C ) , d 3 - 1 \\ if ( {3}^{′} of {rp}_{1} is G or C ) , d 3 - 1 \\ if ( {3}^{′} of {fp}_{2} is G or C ) , d 3 - 1 \\ if ( {3}^{′} of {rp}_{2} is G or C ) , d 3 - 1\end{matrix} ;$$
wherein, f₃ is used for representing the third objective; d3 is an indicative value having a default value equal to 4; a total number of occurrences is initially defined to be zero, and in a condition that the nucleotide type at the 3′ end of any one of the primers is G or C, the total number of occurrences is accumulated by 1; the third objective is calculated by subtracting the total number of occurrences meeting said condition from the default value;
wherein, the fifth objective is defined by the following formula:
$${f}_{5} = {dif}_{Tm} ( {Tm}_{BM} ( {fp}_{1} ) , avgTm ) + {dif}_{Tm} ( {Tm}_{BM} ( {rp}_{1} ) , avg ) + {dif}_{Tm} ( {Tm}_{BM} ( {fp}_{2} ) , avgTm ) + {dif}_{Tm} ( {Tm}_{BM} ( {rp}_{2} ) , avg ) ;$$
$$avgTm = [ {Tm}_{BM} ( {fp}_{1} ) + {Tm}_{BM} ( {rp}_{1} ) + {Tm}_{BM} ( {fp}_{2} ) + {Tm}_{BM} ( {rp}_{2} ) ] \div 4 ;$$
wherein, f₅ is used for representing the fifth objective; Tm_{BM} (fp₁), Tm_{BM}(rp₁), Tm_{BM}(fp₂) and Tm_{BM}(rp₂) each is used for representing the melting temperature of the corresponding one of the first forward primer, the first reverse primer, the second forward primer and the second reverse primer; avgTm is used for representing the average melting temperature;
wherein, the sixth objective is defined by the following formula:
$${f}_{6} = Dimer ( {fp}_{1} , {rp}_{1} ) + Dimer ( {fp}_{1} , {rp}_{2} ) + Dimer ( {fp}_{2} , {rp}_{1} ) + Dimer ( {fp}_{2} , {rp}_{2} ) + Dimer ( {fp}_{1} , {fp}_{2} ) + Dimer ( {rp}_{1} , {rp}_{2} ) + Dimer ( {fp}_{1} , {fp}_{1} ) + Dimer ( {rp}_{1} , {rp}_{1} ) + Dimer ( {fp}_{2} , {fp}_{2} ) + Dimer ( {rp}_{2} , {rp}_{2} ) ;$$
$$Dimer ( {pm}_{a} , {pm}_{b} ) = {Dimer}_{num} ( {pm}_{a} , {pm}_{b} ) - {Dimer}_{user} ;$$
wherein, f₆ is used for representing the sixth objective; Dimerₙᵤₘ(pmₐ,pm_{b}) is used for representing a corresponding binding quantity among identical primers or different primers; Dimerᵤₛₑᵣ is used for representing a user-defined primer biding quantity which is a value showing a binding quantity generated by nucleotides of any two of the primers; Dimer(pmₐ, pm_{b}) is used for representing a level on dimer formation between two of the primers, and is calculated by subtracting the user-defined primer biding quantity from a corresponding binding quantity between any two identical or different primers; Dimer(fp₁, rp₁) represents a level on dimer formation generated between the first forward primer and the first reverse primer; Dimer(fp₁, rp₂) represents a level on dimer formation generated between the first forward primer and the second reverse primer; Dimer(fp₂, rp₁) represents a level on dimer formation generated between the second forward primer and first reverse primer; Dimer(fp₂, rp₂) represents a level on dimer formation generated between the second forward primer and the second reverse primer; Dimer(fp₁, fp₂) represents a level on dimer formation generated between the first forward primer and the second forward primer; Dimer(rp₁, rp₂) represents a level on dimer formation generated between the first reverse primer and the second reverse primer; Dimer(fp₁, fp₁) represents a level on dimer formation generated between two of the first forward primers; Dimer(rp₁, rp₁) represents a level on dimer formation generated between two of the first reverse primers; Dimer(fp₂, fp₂) represents a level on dimer formation generated between two of the second forward primers; Dimer(rp₂, rp₂) represents a level on dimer formation generated between two of the second reverse primers;
wherein, the seventh objective is defined by the following formula:
$${f}_{7} = Hairpin ( {fp}_{1} ) + Hairpin ( {fp}_{2} ) + Hairpin ( {rp}_{2} ) ;$$
$$Hairpin ( primer ) = {Hairpin}_{num} ( primer ) - {Hairpin}_{user} ;$$
wherein, f₇ is used for representing the seventh objective; Hairpinₙᵤₘ(primer) is used for representing a quantity of hairpin structure generated by a corresponding one of the primers; Hairpinᵤₛₑᵣ is used for representing a user-defined primer hairpin structure quantity which is a value showing a self-annealing quantity generated by nucleotides of a primer; Hairpin(primer) is used for representing a corresponding hairpin structure level of one of the primers, and is calculated by subtracting the user-defined primer hairpin structure quantity from a corresponding quantity of hairpin structure generated by a corresponding one of the primers; Hairpin(fp₁) represents a hairpin structure level of the first forward primer; Hairpin(rp₁) represents a hairpin structure level of the first reverse primer; Hairpin(fp₂) represents a hairpin structure level of the second forward primer; Hairpin(rp₂) represents a hairpin structure level of the second reverse primer;
wherein, the eighth objective is defined by the following formula:
$${f}_{8} = {DNA}_{re} ( {fp}_{1} ) + {DNA}_{re} ( {rp}_{1} ) + {DNA}_{re} ( {fp}_{2} ) + {DNA}_{re} ( {rp}_{2} ) ;$$
wherein, f₈ is used for representing the eighth objective; DNAᵣₑ(fp₁), DNAᵣₑ(rp₁), DNAᵣₑ(fp₂) and DNAᵣₑ(rp₂) are respectively used for representing a frequency re-appearing in the DNA template segment of the first forward primer, the first reverse primer, the second forward primer and the second reverse primer;
wherein, the tenth objective is defined by the following formula:
$${f}_{10} = \{ \begin{matrix}d 10 = 3 \\ if {pl}_{1} \geq 100 , d 10 - 1 \\ if {pl}_{2} \geq 100 , d 10 - 1 \\ if {pl}_{3} \geq 100 , d 10 - 1\end{matrix} ;$$
wherein, f₁₀ is used for representing the tenth objective; d10 is an indicative value having a default value equal to 3 and is used for calculating the overall conformity level among product lengths; a total number of occurrences is initially defined to be zero, and in a condition that any to-be-evaluated product length is larger than or equal to 100, the a total number of occurrences is accumulated by 1; the tenth objective is calculated by subtracting the total number of occurrences meeting said condition from the default value; said to-be-evaluated product lengths are respectively defined by the first length, the second length and the third length.

4. The multi-objective design method for confronting two-pair primers as claimed in claim 3, wherein the design objective further includes the eighth objective.

5. The multi-objective design method for confronting two-pair primers as claimed in claim 3, wherein the design objective further includes the sixth objective and the seventh objective.

6. The multi-objective design method for confronting two-pair primers as claimed in claim 3, wherein the design objective further includes the sixth objective, the seventh objective and the eighth objective.

7. The multi-objective design method for confronting two-pair primers as claimed in claim 3, wherein the design objective further includes the first objective, the second objective, the third objective, the fifth objective, the sixth objective, the seventh objective, the eighth objective and the tenth objective.

8. The multi-objective design method for confronting two-pair primers as claimed in claim 1, wherein in the calculation means, a Pareto Chart Analysis is applied to obtain the optimal solution.

9. The multi-objective design method for confronting two-pair primers as claimed in claim 4, wherein in the calculation means, a Pareto Chart Analysis is applied to obtain the optimal solution.

10. The multi-objective design method for confronting two-pair primers as claimed in claim 5, wherein in the calculation means, a Pareto Chart Analysis is applied to obtain the optimal solution.

11. The multi-objective design method for confronting two-pair primers as claimed in claim 6, wherein in the calculation means, a Pareto Chart Analysis is applied to obtain the optimal solution.

12. The multi-objective design method for confronting two-pair primers as claimed in claim 7, wherein in the calculation means, a Pareto Chart Analysis is applied to obtain the optimal solution.

13. The multi-objective design method for confronting two-pair primers as claimed in claim 8, wherein the calculation means is a Guided Population Archive Whale Optimization Algorithm.

14. The multi-objective design method for confronting two-pair primers as claimed in claim 9, wherein the calculation means is a Guided Population Archive Whale Optimization Algorithm.

15. The multi-objective design method for confronting two-pair primers as claimed in claim 10, wherein the calculation means is a Guided Population Archive Whale Optimization Algorithm.

16. The multi-objective design method for confronting two-pair primers as claimed in claim 11, wherein the calculation means is a Guided Population Archive Whale Optimization Algorithm.

17. The multi-objective design method for confronting two-pair primers as claimed in claim 12, wherein the calculation means is a Guided Population Archive Whale Optimization Algorithm.
